(19) United States
(12) Patent Application Publication
(10) Pub. No.: US 2026/0055452 A1
(43) Pub. Date: Feb. 26, 2026

(54) MODIFIED NUCLEOTIDES AND RELATED METHODS FOR NANOPORE SEQUENCING

(71) Applicant: Illumina, Inc., San Diego, CA (US)

(72) Inventors: Jeffrey G. Mandell, Rancho Santa Fe, CA (US); Jessica Killian, San Diego, CA (US); Tsz Ying Yuen, Singapore (SG); Abdul Sadeer Abd Salam, Singapore (SG); Ramesh Neelakandan, Singapore (SG); Boyan Boyanov, San Diego, CA (US); Xiangyuan Yang, Singapore (SG); John Moon, Rancho Santa Fe, CA (US); Yin Nah Teo, Singapore (SG)

(21) Appl. No.: 19/305,635

(22) Filed: Aug. 20, 2025

Related U.S. Application Data
(60) Provisional application No. 63/685,610, filed on Aug. 21, 2024.

(51) Int. Cl. C12Q 1/6869

(52) U.S. Cl. CPC C12Q 1/6869

(57) ABSTRACT

Disclosed herein is a modified oligonucleotide, the modified oligonucleotide comprises one or more modifications in at least one of a phosphate backbone, a nucleobase, or a sugar. The one or more modifications impede translocation through a nanopore by increasing non-covalent interactions with the interior of the nanopore or the lipid bilayer that supports the nanopore, increasing the bulk size or the steric hinderance of oligonucleotide, or alter the charge density of the oligonucleotide.

### RELATED APPLICATIONS

This application claims priority to U.S. Provisional Patent Application No. 63/685,610, filed Aug. 21, 2024, which is hereby incorporated by reference in its entirety.

### BACKGROUND

Some polynucleotide sequencing techniques involve performing a large number of controlled reactions on support surfaces or within predefined reaction chambers. The controlled reactions may then be observed or detected, and subsequent analysis may help identify properties of the polynucleotide involved in the reaction. Examples of such sequencing techniques include next-generation sequencing or massive parallel sequencing involving sequencing-by-ligation, sequencing-by-synthesis, reversible terminator chemistry, or pyrosequencing approaches.

Some polynucleotide sequencing techniques utilize a nanopore, which can provide a path for an ionic electrical current. For example, as the polynucleotide traverses through the nanopore, it influences the electrical current through the nanopore. Each passing nucleotide, or series of nucleotides, that passes through the nanopore yields a characteristic blockage current. These characteristic electrical currents of the traversing polynucleotide can be recorded to determine the sequence of the polynucleotide.

### SUMMARY

Provided in examples herein are modified nucleotides and oligonucleotides, methods for sequencing biopolymers, particularly polynucleotides, and systems and kits for performing the methods.

The systems, kits, and methods disclosed herein each have several aspects, no single one of which is solely responsible for their desirable attributes. Without limiting the scope of the claims, some prominent features will now be discussed briefly. Numerous other examples are also contemplated, including examples that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

Additional details of exemplary nanopore sequencing devices which can be used with the disclosed technology, and methods of operating the devices, can be found in U.S. patent application Ser. Nos. 18/003,493 and 18/003,883, the entirety of each of the disclosures is incorporated herein by reference.

Disclosed herein is a modified oligonucleotide, the modified oligonucleotide comprises one or more modifications in at least one of a phosphate backbone, a nucleobase, or a sugar, wherein the one or more modifications impede translocation through a nanopore.

In some embodiments, the one or more modifications are synthetic moieties to: (a) increase non-covalent interactions with an interior of the nanopore, (b) increase the bulk size of the oligonucleotide, (c) alter the charge density of the oligonucleotide, (d) increase the steric hinderance of the oligonucleotide, and/or (e) increase non-covalent interactions with a lipid bilayer that supports the nanopore.

In some embodiments, the one or more modifications introduces chirality to a non-bridging oxygen on the phosphate backbone.

In some embodiments, the one or more modifications replaces a single bridging oxygen in the phosphate backbone with one or more elements selected from O, S, and —NH—.

In some embodiments, the nucleobase comprises a nucleobase modification including one or more of: glutamic acid, polyglutamic acid, polylysine, polyamines, fluorine, G-quadraplexes, DNA origami, or macrocycles.

In some embodiments, the modified oligonucleotide additionally comprises a modified nucleotide containing a cyclic loop bridging the nucleobase and the phosphate backbone or bridging one portion of the phosphate backbone and an oxygen on the phosphate backbone.

Disclosed herein is a modified oligonucleotide having one of the following structures:

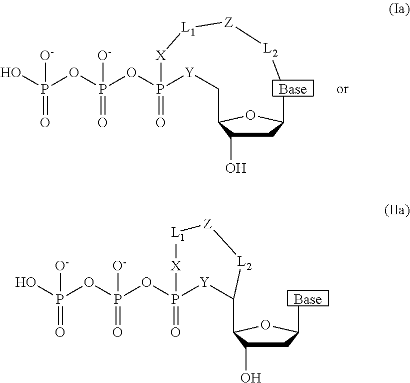

Wherein X is —O—, —CH₂—, or —NH—; Y is —O—, —S—, or —NH—; Base is a nucleobase; L₁ is a first linking group; L₂ is a second linking group; and Z is a cyclic loop modification containing at least one moiety to reduce, slow, or halt translocation of the nucleotide through a nanopore.

In some embodiments, Z includes a spacer and reporter, wherein the spacer and reporter each comprises one or more of the following moieties: (a) polypeptides having 10 to 100 repeating units, (b) pseudopeptides having 10 to 100 repeating units, (c) hydrophilic polymers having 10 to 100 repeating units, and (d) hydrophobic polymers having 10 to 100 repeating units.

In some embodiments, Z includes one or more moieties to increase non-covalent interaction with the interior of the nanopore.

In some embodiments, Z includes one or more moieties to alter or reduce the net charge of the modified nucleotide.

In some embodiments, Z includes one or more moieties to increase the bulk size of Z and increase steric hinderance.

In some embodiments, each of L₁ and L₂ independently comprises a conjugating moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene.s

Disclosed herein is a method for determining a sequence of a polynucleotide in a nanopore-based sequencing system, the method comprises providing a modified oligonucleotide, wherein the modified oligonucleotide comprises one or more modifications in at least one of a phosphate backbone, a nucleobases, or a sugar, wherein the one or more modifications impede translocation through a nanopore. In some embodiments, the method comprises applying a bias across a nanopore to cause the modified oligonucleotide to insert into and translocate through the nanopore. In some embodiments, the method comprises detecting and identifying a reporter moiety when the modified oligonucleotide passes through the nanopore. In another aspect, the method comprises detecting and identifying a base on the oligonucleotide when the oligonucleotide passes through the nanopore.

In some embodiments, the modified oligonucleotide comprises one or more modified nucleotides that comprise a cyclic loop, the cyclic loop having a first end attached to a first position of a nucleotide of the oligonucleotide and a second end attached to a second position of the nucleotide.

In some embodiments, the method further comprises cleaving a cleavable bond on the one or more modified nucleotides between the first and the second positions, thereby elongating the one or more modified nucleotides to form an elongated polymer, and (i) detecting and identifying a reporter moiety when the elongated polymer passes through the nanopore; or (ii) detecting and identifying a nucleobase on the one or more nucleotides when the elongated polymer passes through the nanopore.

In some embodiments, the cyclic loop comprises a first linking group, a second linking group, and a spacer between the first and the second linking groups.

In some embodiments, the spacer comprises a oligonucleotide or modified oligonucleotide or phosphoramidite analogs having 10 to 100 repeating units, polypeptide having 10 to 100 repeating units, alkyl chains having 5 to 50 carbons, hydrophilic polymers having 10 to 100 repeating units selected form the group consisting of polyethyleneglycol, polyvinyl alcohol, polyacrylamide, polyvinylpyrrolidone, polystyrenesulfonate, and polyethyleneimine, hydrophobic polymers having 10 to 100 repeating units selected from the group consisting of polylactic acid, polymethylmethacrylate, and polystyrene, and combinations thereof.

In some embodiments, the one or more modifications in at least one of a phosphate backbone, a nucleobase, and a sugar are isolated in a single region of the oligonucleotide.

In another aspect, the one or more modifications in at least one of a phosphate backbone, a nucleobase, and a sugar are interspersed between one or more modified nucleotides in the modified oligonucleotide.

Disclosed herein are kits for performing the disclosed methods as provided. The kit may comprise nucleotides or oligonucleotides with any of the modifications described in the present disclosure.

Disclosed herein are systems for determining a sequence of a polynucleotide using any of the methods described in the present disclosure. In another aspect, the polynucleotide comprises a plurality of oligonucleotides with any of the modifications described in the present disclosure.

Disclosed herein is a modified nucleotide, the modified nucleotide comprises a cyclic loop linking one or more atoms of the modified nucleotide, wherein the cyclic loop comprises a first linking group, a second linking group, and optionally, a spacer and reporter. In some embodiments, the spacer and reporter each comprise one or more of the following moieties: (a) polypeptides having 10 to 100 repeating units, (b) pseudopeptides having 10 to 100 repeating units, (c) hydrophilic polymers having 10 to 100 repeating units, and (d) hydrophobic polymers having 10 to 100 repeating units.

In some embodiments, the cyclic loop further comprises nucleosidic moieties, non-nucleosidic moieties, and/or peptide moieties.

In some embodiments, the modified nucleotide further comprises one or more of the following: backbone modification(s), sugar modification(s), and base modification(s) configured to reduce the translocation speed of the modified nucleotide through a nanopore having a constriction zone diameter less than 5 nm in width.

Disclosed herein is an oligonucleotide strand, the oligonucleotide strand comprises any of the modified nucleotides described in the present disclosure.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 illustrates three diastereomers modelled using MM2 energy minimization simulation to illustrate the different conformation adopted by each of the diastereomers.

FIG. 2 illustrates some embodiments where one or more modifications replaces a single bridging oxygen in the phosphate backbone with one or more elements (X or Y) selected from O, S, and —NH—.

FIG. 3 illustrates some embodiments where a modified nucleotide comprises a speed control element interspersed in a cyclic loop.

FIG. 4 illustrates some embodiments where a modified nucleotide comprises a speed control element isolated within a specific region of a cyclic loop.

FIG. 5 illustrates some embodiments where a modified k-mer comprises a speed control element interspersed in a cyclic loop.

FIG. 6 illustrates some embodiments where a modified k-mer comprises a speed control element isolated within a specific region of a cyclic loop.

FIG. 7 illustrates some embodiments where one or more moieties comprises

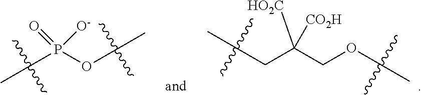 [SMILES: *OP(*)(=O)[O-] ; *CC(CO*)(C(=O)O)C(=O)O]

FIG. 8 illustrates some embodiments where the one or more moieties is

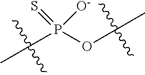 [SMILES: *OP(*)([O-])=S]

and the nucleotide comprises locked nucleic acid modifications.

FIG. 9 illustrates some embodiments where the one or more moieties are

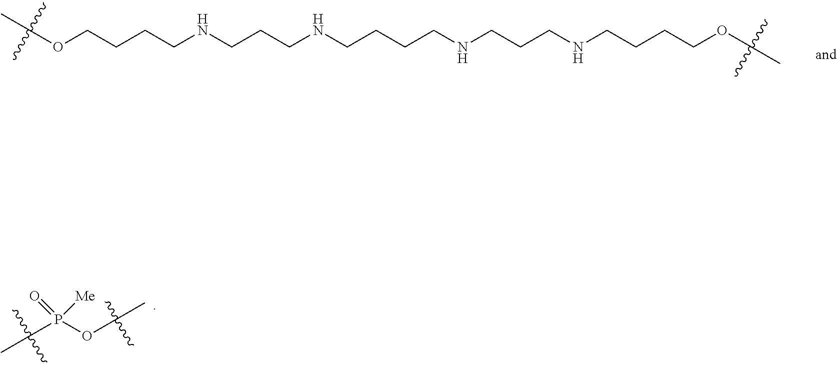 [SMILES: *OCCCCNCCCNCCCCNCCCNCCCCO* ; *OP(*)(C)=O]

FIG. 10 illustrates some embodiments where the one or more moieties is

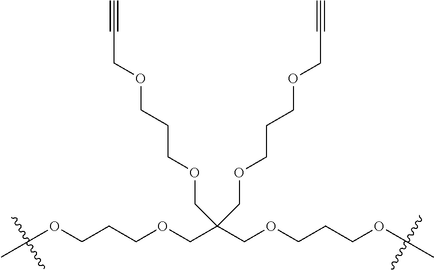 [SMILES: *OCCCOCC(COCCCO*)(COCCCOCC#C)COCCCOCC#C]

and the nucleotide comprises locked nucleic acid modifications.

FIG. 11 illustrates some embodiments where the alkyne on the one or more moieties is further functionalized via Cu-click to increase steric bulk along the cyclic loop.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the term “modified oligonucleotide” refers to a polymeric chain of nucleobases or nucleotides assembled with moieties comprising a modified nucleobase, modified sugar rings (e.g. LNA, constraint ethyl, ethylene bridged, TNA, 2′-Ome, 2′F, 2′-MOE) or nucleobases attached to a scaffold (e.g. unlock, 4′-thio, CeNA, HNA, TNA, GNA, FNA).

As used herein, the term “phosphoramidite analogs” refers to any polymer synthesized using phosphoramidite or related chemistries resulting in the formation of phosphodiester, methylphosphonate, or phosphorothioate bonds between each moiety.

As used herein, the term “nanopore” is intended to mean a hollow structure discrete from, or defined in, and extending across the membrane. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage (through a nanoscale opening extending through the nanopore structure) of the ions or water-soluble molecules from one side of the membrane to the other side of the membrane. The diameter of the nanoscale opening extending through the nanopore structure can vary along its length (i.e., from one side of the membrane to the other side of the membrane), but at any point is on the nanoscale (i.e., from about 1 nm to about 100 nm, or to less than 1000 nm). Examples of the nanopore include, for example, biological nanopores, solid-state nanopores, and biological and solid-state hybrid nanopores. In some embodiments, a nanopore refers to a pore having an opening with a diameter at its most narrow point of about 0.3 nm to about 2 nm. For example, a nanopore may be a solid-state nanopore, a graphene nanopore, an elastomer nanopore, or may be a naturally-occurring or recombinant protein that forms a tunnel upon insertion into a bilayer, thin film, membrane, or solid-state aperture, also referred to as a protein pore or protein nanopore herein (e.g., a transmembrane pore). If the protein inserts into the membrane, then the protein is a tunnel-forming protein.

As used herein, the term “diameter” is intended to mean a longest straight line inscribable in a cross-section of a nanoscale opening through a centroid of the cross-section of the nanoscale opening. It is to be understood that the nanoscale opening may or may not have a circular or substantially circular cross-section (the cross-section of the nanoscale opening being substantially parallel with the cis/trans electrodes). Further, the cross-section may be regularly or irregularly shaped.

As used herein, “cis” refers to the side of a nanopore opening through which an analyte or modified analyte enters the opening or across the face of which the analyte or modified analyte moves.

As used herein, “trans” refers to the side of a nanopore opening through which an analyte or modified analyte (or fragments thereof) exits the opening or across the face of which the analyte or modified analyte does not move.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, a “moiety” is one of two or more parts into which something may be divided, such as, for example, the various parts of a tether, a molecule or a probe.

As used herein, a “reporter” is composed of one or more reporter elements or reporter moieties. Reporters include what are known as “tags” and “labels.” The linker construct (when including reporter moiety) or nucleobase residue of the elongated polymer can be considered a reporter. Reporters serve to parse the identity of the target nucleic acid. Reporters may include constituent sub-reporters, and multiple reporters may be present on a single nucleotide. When present in the readhead of a nanopore, reporters provide distinctive and sometimes unique blockage currents at given read voltages.

As used herein, a “linker” is a molecule or moiety that joins two molecules or moieties and provides spacing between the two molecules or moieties such that they are able to function in their intended manner. For example, a linker can comprise a diamine hydrocarbon chain that is covalently bound through a reactive group on one end to an oligonucleotide analog molecule and through a reactive group on another end to a solid support, such as, for example, a bead surface. Coupling of linkers to nucleotides and substrate constructs of interest can be accomplished through the use of coupling reagents that are known in the art (see, e.g., Efimov et al., Nucleic Acids Res. 27:4416-4426, 1999). Methods of derivatizing and coupling organic molecules are well known in the arts of organic and bioorganic chemistry. A linker may also be cleavable or reversible.

As used herein, the term “polypeptide nanopore” is intended to mean a protein/polypeptide that extends across the membrane, and permits ions, electric current, polymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, an α-helix bundle nanopore and a β-barrel nanopore. Example polypeptide nanopores include α-hemolysin, Mycobacterium smegmatis porin A (MspA), gramicidin A, maltoporin, OmpF, OmpC, PhoE, Tsx, F-pilus, etc. The protein α-hemolysin is found naturally in cell membranes, where it acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore.

As used herein, a “peptide” refers to two or more amino acids joined together by an amide bond (that is, a “peptide bond”). Peptides comprise up to or include 50 amino acids. Peptides may be linear or cyclic. Peptides may be α, β, γ, δ, or higher, or mixed. Peptides may comprise any mixture of amino acids as defined herein, such as comprising any combination of D, L, α, β, γ, δ, or higher amino acids.

As used herein, a “protein” refers to an amino acid sequence having 51 or more amino acids.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

The nanopores disclosed herein may be hybrid nanopores. A “hybrid nanopore” refers to a nanopore including materials of both biological and non-biological origins. An example of a hybrid nanopore includes a polypeptide-solid-state hybrid nanopore and a polynucleotide-solid-state nanopore.

The application of the electric potential difference across a nanopore may force the translocation of a nucleic acid through the nanopore. One or more signals are generated that correspond to the translocation of the nucleotide through the nanopore. Accordingly, as a target polynucleotide, or as a mononucleotide or a probe derived from the target polynucleotide or mononucleotide, transits through the nanopore, the current across the membrane changes due to base-dependent (or probe dependent) blockage of the constriction, for example. The signal from that change in current can be measured using any of a variety of methods. Each signal is unique to the species of nucleotide(s) (or linker constructs with a reporter moiety region) in the nanopore, such that the resultant signal can be used to determine a characteristic of the polynucleotide. For example, the identity of one or more species of nucleotide(s) (or probe) that produces a characteristic signal can be determined.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothiolate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, voltage, conductance, or a transverse electrical effect. An “electronic current” or “electric current” refers to a flow of electric charge. In an example, an electrical signal may be an electric current passing through a nanopore, and the electric current may flow when an electric potential difference is applied across the nanopore.

As used herein, the term “driving force” is intended to mean an electrical current that allows a polynucleotide to translocate through the nanopore. In some embodiments, the electrical current may flow when an electric potential difference is applied across the nanopore.

As used herein, the term “holding force” is intended to mean a resistance that slows and/or stops a polynucleotide to translocate through the nanopore. In some embodiments, the holding force is overcome by the application of a driving force. Thus, the driving force overcomes/overrides the resistance that slows and/or stops a polynucleotide, thereby allowing the polynucleotide to translocate through the nanopore.

As used herein, the term “modification” is intended to mean a moiety attached to a nucleotide. A modification may provide a resistance (in the form of a “holding force”) that slows and/or stops a polynucleotide to translocate through the nanopore unless the resistance due to the modification is overcome by a “driving force.” The resistance provided by the modification is due to a property of the modification (e.g., size, geometry, and/or non-covalent interaction with the nanopore). Modifications can operate as a ratchet or a brake for the polynucleotide translocation through a nanopore. A modification can be attached to any part of the nucleotide and can also be attached to the nucleotide at two locations forming a loop. The modification may also be referred to as an arresting construct.

The aspects and examples set forth herein and recited in the claims can be understood in view of the above definitions.

### Overview

Nanopore sequencing has been demonstrated as a viable method for parallel, real-time sequencing of a polynucleotide, such as DNA and RNA. In strand-based nanopore sequencing approaches, a sequence is determined by reading the base-specific changes in ionic current that are produced as a strand of polynucleotide passes through the constriction of a nanopore. However, using nanopores to sequence depends heavily on the size of the nanopore constriction, and the rate of translocation of polynucleotide through the nanopore.

A common drawback with nanopore sequencers is that the nanopore is sensitive to multiple bases of a polynucleotide strand in the nanopore, as opposed to reading single base one at a time. The nanopore reads a k-mer, complicating the deconvolution process. For instance, for a k-mer of 5 bases, the number of possible signals is 4\hat{ }5=1,024. The speed of translocation of natural single stranded polynucleotide is in the order of >10 million nucleotides per second, way above the rate that is compatible with electronics and detector. Fast translocation speed directly impacts accuracy either due to incompatibility with the detector resulting in deletion errors or skipped bases arising from low dwell time of the polynucleotide analyte.

A polynucleotide has significant interactions with protein, in particular between the phosphate backbone of polynucleotide with the side chains of amino acid residues. In fact, proteins are able to interact with a large variety of ligands or molecules through non-covalent interactions such as hydrophobic, hydrogen bonding, pi-stacking, salt bridges and amide stacking. The MspA nanopore is a protein-based pore. Therefore, enhancing the non-covalent interactions of the polynucleotide analyte with MspA residues would be feasible as a mean of controlling the translocation speed. In addition, the narrow constriction zone of MspA would allow other methods of slowing translocation to be feasible, for example the introduction of bulky or rigid modification. Another advantage of controlling the translocation speed would be to achieve a tighter gamma distribution of events.

### Speed Control Elements in Modified Oligonucleotides

Disclosed herein is a modified oligonucleotide, the modified oligonucleotide comprises one or more modifications in at least one of a phosphate backbone, a nucleobase, or a sugar, wherein the one or more modifications impede translocation through a nanopore. In some embodiments, the one or more modifications are synthetic moieties to: (a) increase non-covalent interactions with an interior of the nanopore, (b) increase the bulk size of the oligonucleotide, (c) alter the charge density of the oligonucleotide, and/or (d) increase the steric hinderance of the oligonucleotide, and/or (e) increase non-covalent interactions with a lipid bilayer that supports the nanopore.

In some embodiments, the one or more modifications are introduced through enzymatic means (polymerase incorporation, ligase ligation, etc), or chemical means such as automated polynucleotide synthesis with modified amidites. Examples of enzymatic incorporation are described in, but not limited to, Nature Chem. 2019, 11, 533; Nucleosides and Nucleotides 1995, 14, 91. Examples of chemically synthesis are describe in, not limited to, Nucleic Acids Res. 2019, 47, 5465; PNAS 2020, 117, 32370; Org Biomol Chem. 2012, 10, 746; New J. Chem. 2019, 43, 4323; PNAS 2002, 99, 15965; Front. Chem. 2021, 9, 619052.

### Backbone Modifications

In a native polynucleotide, such as DNA or RNA, the phosphodiester backbone is non-chiral and carries a single net negative charge per phosphodiester group, imparting an overall negative charge density across the entire polynucleotide strand. This key feature allows the polynucleotide strand to be responsive to an externally applied electric field, in which voltage driven electrophoretic migration is the main driving force for polynucleotide translocation through nanopores.

In some embodiments, modifications may be introduced into the backbone of the polynucleotide. The one or more modifications may increase non-covalent interactions with the pore interior. In some embodiments, the one or more modifications remove the negative charge and reduce the overall charge density of the polynucleotide, directly affecting the magnitude of the electrophoretic force acting on the polynucleotide. In some embodiments, the one or more modifications introduce stereogenic phosphorus centers which affect the structural configuration adopted by the polynucleotide. In some embodiments, the one or more modifications introduce bulky moieties. In some embodiments, the bulky moieties reduce flexibility of the polynucleotide strand and increase friction with the pore interior.

In some embodiments, the one or more modifications replace a non-bridging oxygen on the phosphate backbone. For example, as seen in the modified oligonucleotide structure below, a non-bridging oxygen is replaced by a R group (i.e., modification).

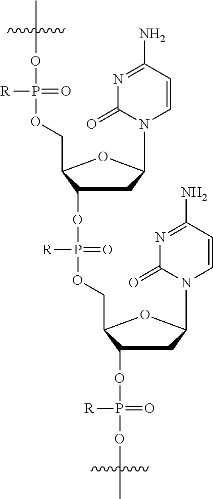

The modification (represented by R) may include an alkyl chains an aromatic group, a heterocycle, a heteroatom, a combination thereof, or other bulky substituents. In some embodiments, the modification/R may be selected from the group consisting of:

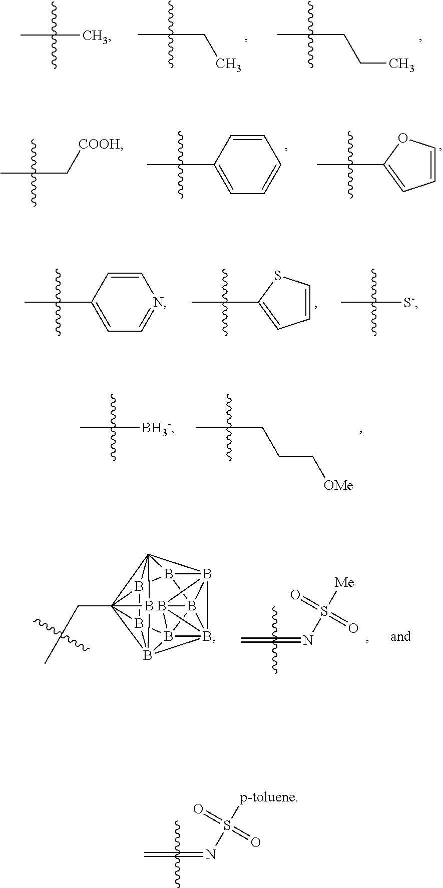

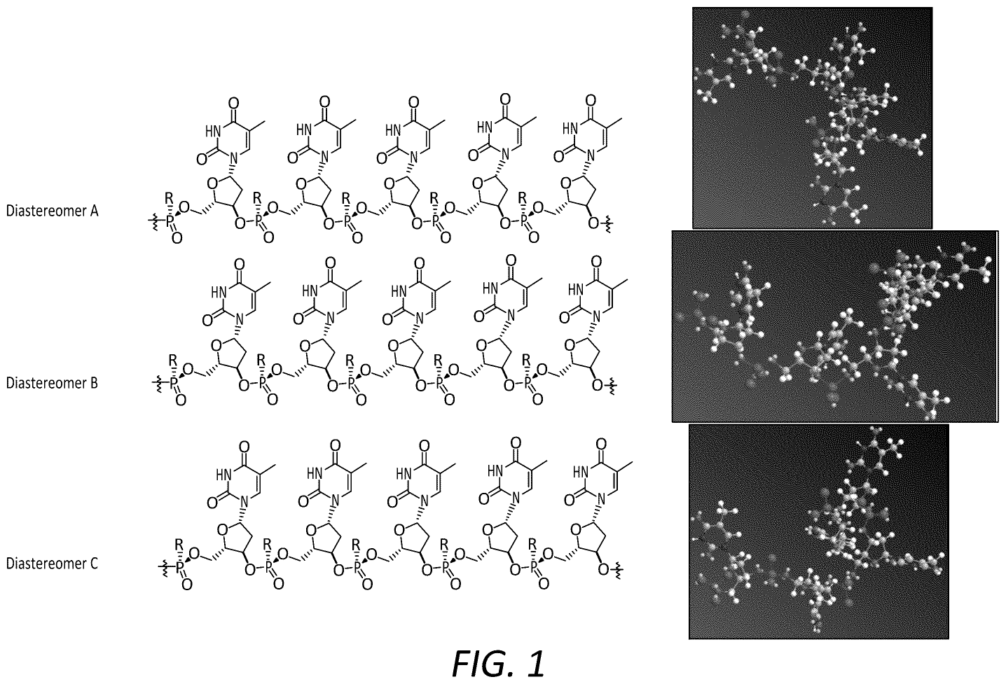

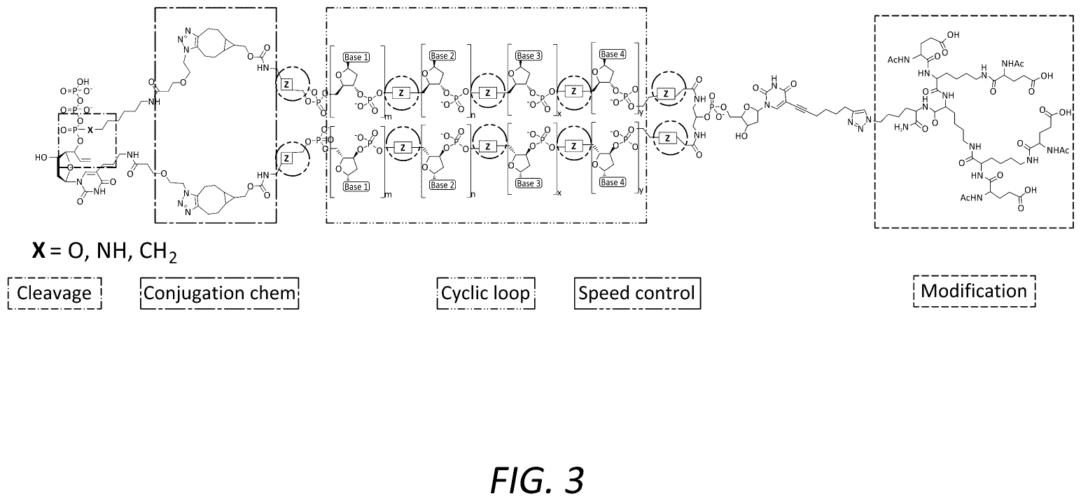

In some embodiments, substituting the R group at the non-bridging position (FIG. 3) has various effects. For example, the modification may increase non-covalent interactions with the pore interior. In some instances, the modification may remove the negative charge and reduce overall charge density of the polynucleotide. This may directly affect eh magnitude of the electrophoretic force acting on the polynucleotide. In some embodiments, the modification may introduce stereogenic phosphorus centers, which affects the structural configuration adopted by the polynucleotide. FIG. 1 illustrates three diastereomers modelled using MM2 energy minimization simulation to illustrate the different conformation adopted by each of the diastereomers. These conformations either restrict or encourage the translocation of each diastereomer through the narrow constriction of a nanopore. In some embodiments, the modifications may include bulky moieties that reduce the flexibility of the polynucleotide strand and increase friction with the pore interior.

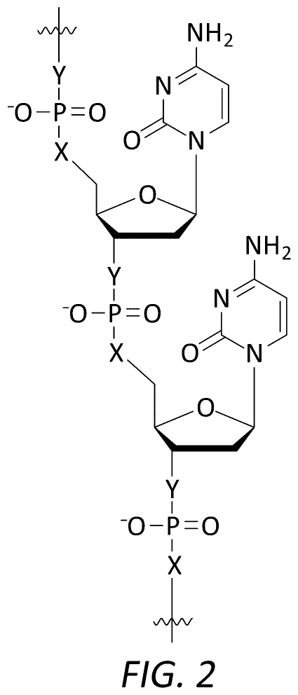

In some embodiments, the one or more modifications replaces a single bridging oxygen in the phosphate backbone with one or more elements selected from O, S, and —NH—. (FIG. 2). In some embodiments, X and Y may independently be S, O, or NH. In some embodiments, X is NH and Y is O. In some embodiments, X is O and Y is NH or S. In some embodiments, X is NH and Y is O. In some embodiments X is S and Y is O. In some embodiments, X is O and Y is S These backbones may be prepared by enzymatic incorporation with modified triphosphate nucleotides or by chemical synthesis methods.

In some embodiments, the phosphate group can be replaced entirely with a chemically modified backbone. In some embodiments, the chemically modified backbone comprises a triazole. In some embodiments, the one or more modifications comprises a substitution on the phosphate backbone with one or more nucleotide linkers to produce a modified oligonucleotide having one of the following nucleotide linking structures:

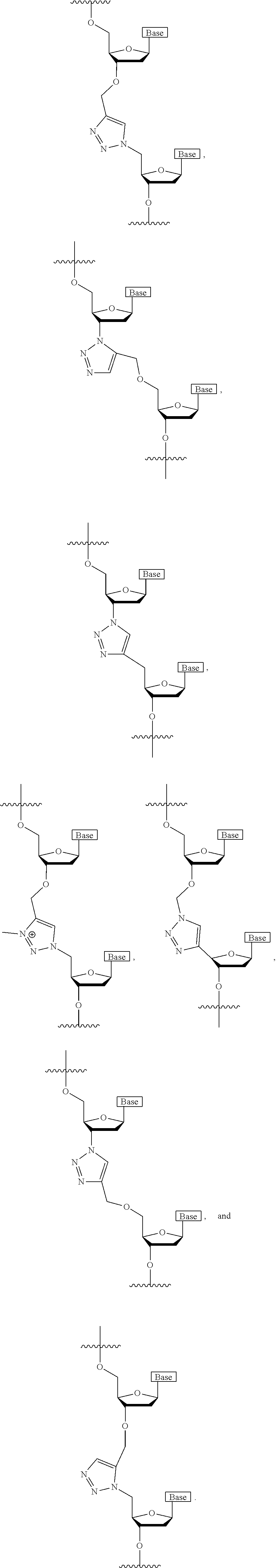

### Sugar Modifications

In some embodiments, the one or more modifications comprise a modified sugar structure. In some embodiments, modifying the sugar structure presents an opportunity to tune non-covalent interactions with the pore interior. In some embodiments, modifying the sugar structure presents an opportunity to introduce additional stereogenic centers to modify the structural configuration adopted by the oligonucleotide. In some embodiments, modifying the sugar structure presents an opportunity to increase steric bulk which reduces the flexibility of the oligonucleotide/polynucleotide and/or increase friction with the pore interior.

In some embodiments the one or more modifications comprises a modified sugar structure and the oligonucleotide contains a modified nucleotide selected from the group consisting of:

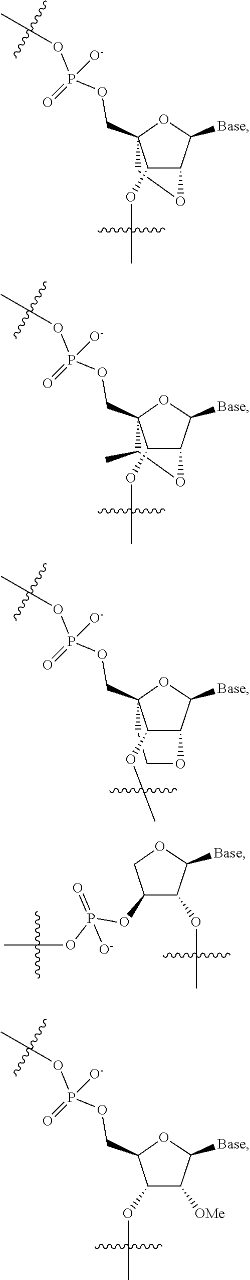

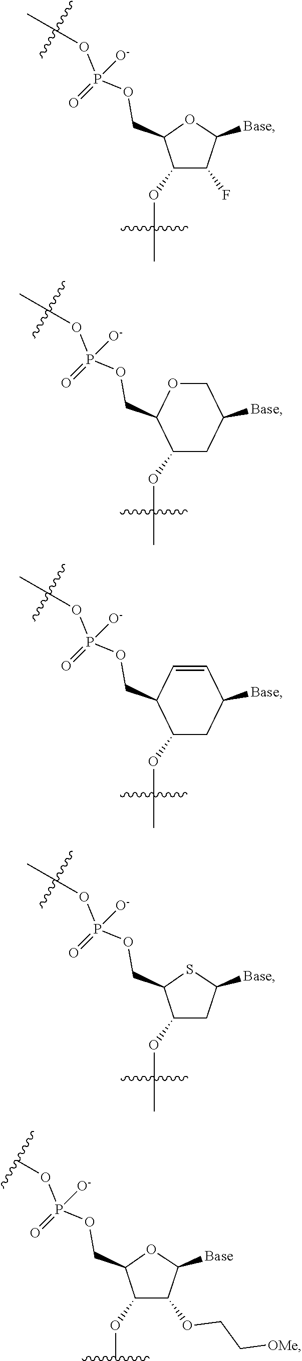

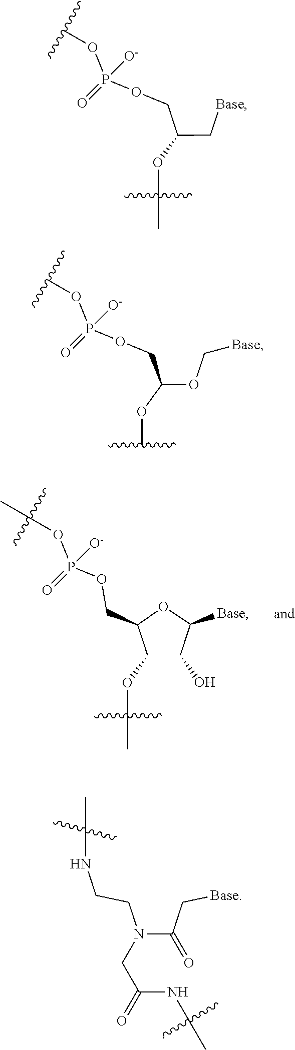

Numerous types of sugar modified nucleic acids have been reported in literature (see Front. Mol. Biosci. 2019, 6, 28 and Current Opinion in Biotechnology 2019, 60, 259). Many of these modified nucleic acids have been used to prepare modified oligomers either enzymatically or synthetically.

### Base Modifications

In some embodiments, the one or more modifications comprise a modified nucleobase structure. A plethora of molecules can be easily attached to the base. In some embodiments, a moiety is attached to the nucleobase by click chemistry. In some embodiments, click chemistry is performed with a copper catalyst. Other conjugation reactions could also be considered (Bioconjugate Chem. 2021, 32, 63).

In some embodiments, the one or more modifications tune non-covalent interactions

with the pore interior. In some embodiments, the one or more modifications tune overall charge density of the polynucleotide, directly affecting the magnitude of the electrophoretic force acting on the polynucleotide. In some embodiments, the one or more modifications introduce bulky moieties. In some embodiments, the bulky moieties reduce flexibility of the polynucleotide strand and increase friction with the pore interior. In some embodiments, the one or more modifications tune interactions with a membrane (e.g., lipid bilayer).

In some embodiments, the modified nucleobase is selected from the group consisting of:

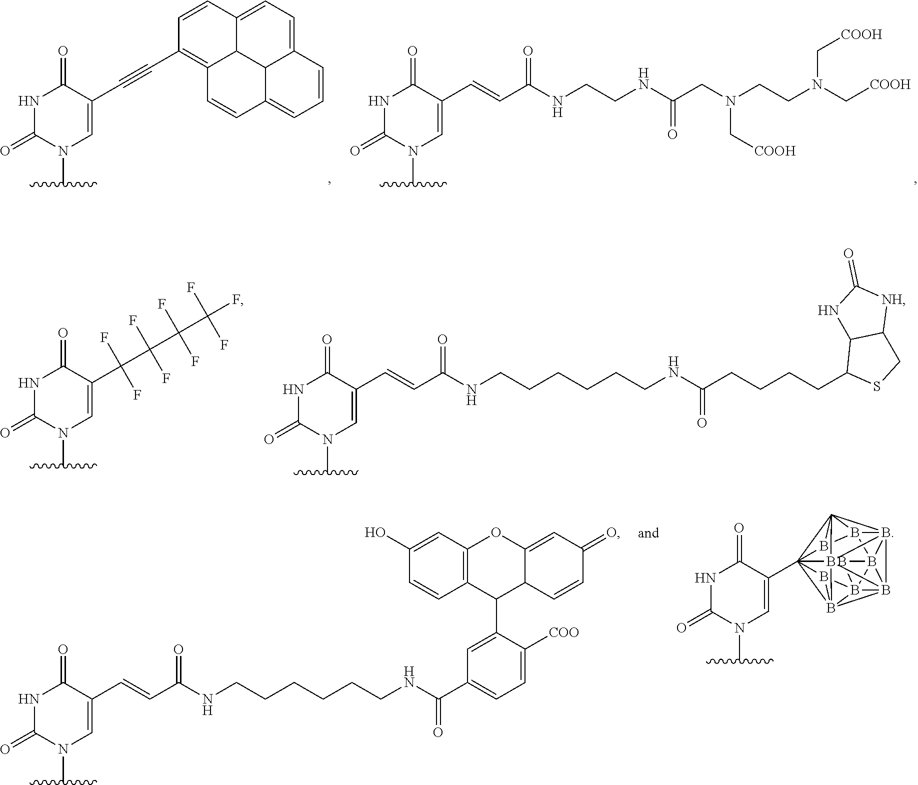

Base modifications are aa robust category of modifications because they are easily accessed by chemical methods (e.g., via click conjugation to alkyne-modification). Furthermore, base modifications are well tolerated by enzymes such as polymerases.

In some embodiments, the one or more modifications comprises a moiety attached to the nucleobase and the moiety is selected from the group consisting of:

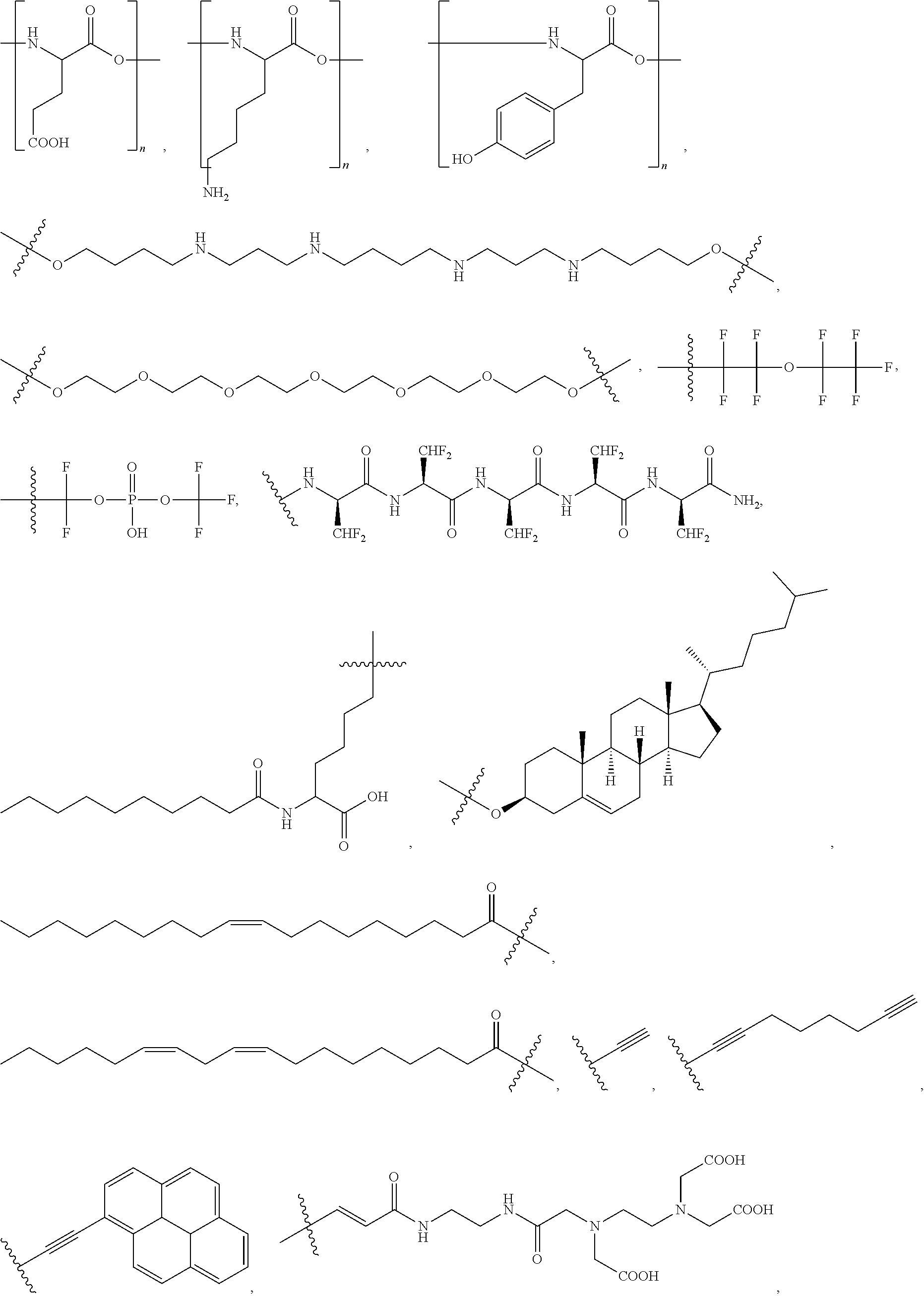

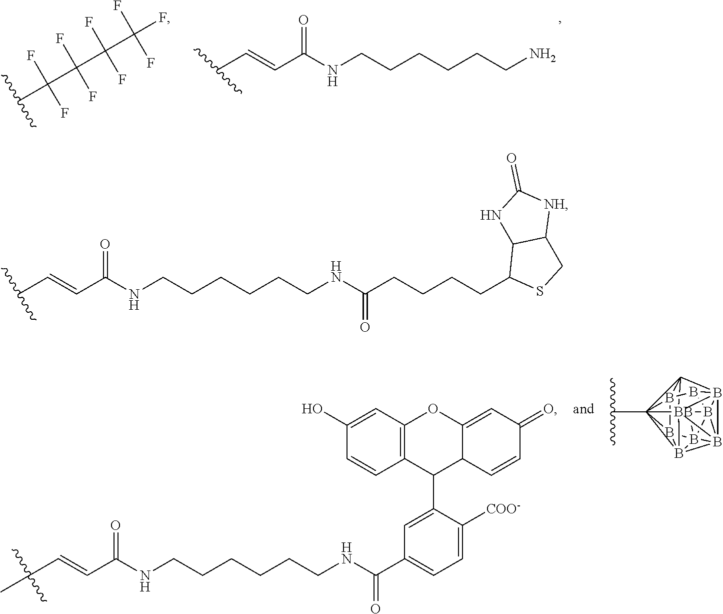

wherein n is a positive integer. In some embodiments, n is an integer selected from 1 to 100.

Depending on the desired properties, the modifications can be tuned to achieve certain structural features. In some embodiments, the negative charged density of the oligonucleotide can be increased by introducing negative charges such as polyglutamic acid. In some embodiments, the negative charge density of the oligonucleotide can be reduced by introducing polylysine or polyamines (e.g., spermine). In some embodiments, the one or more modifications comprise fluorine moieties that encourage the interactions between a lipid bilayer and the oligonucleotide. In some embodiments, the nucleobase comprises a nucleobase modification including one or more of: glutamic acid, polyglutamic acid, polylysine, polyamines, fluorine, G-quadraplexes, DNA origami, and macrocycles.

### Speed Control Elements in Cyclic Loop Nucleotide or K-mer

In some embodiments, the modified oligonucleotide additionally comprises one or more modified nucleotides containing a cyclic loop bridging the nucleobase and the phosphate backbone or bridging one portion of the phosphate backbone and an oxygen on the phosphate backbone. In some embodiments, the cyclic loop may connect two nucleotides that are adjacent to each other, every other nucleotides, or every third nucleotides in the polynucleotide sequence.

In some embodiments, a modified nucleotide has one of the following structures:

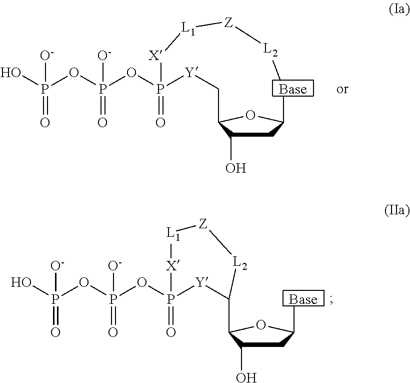

wherein X′ is —O—, —CH₂—, or —NH—; Y′ is —O—, —S—, or —NH—; Base is a nucleobase; L₁ is a first linking group; L₂ is a second linking group; and Z is a cyclic loop modification containing at least one moiety to reduce, slow, or halt translocation of the polynucleotide through a nanopore.

In some embodiments, Z includes a spacer, reporter, and a speed control element, wherein the spacer and reporter each comprises one or more of the following moieties: (a) polypeptides having 10 to 100 repeating units, (b) pseudopeptides having 10 to 100 repeating units, (c) hydrophilic polymers having 10 to 100 repeating units, and (d) hydrophobic polymers having 10 to 100 repeating units, and the speed control element may be a moiety that can also slow or halt the translocation of the polynucleotide that contains the modified nucleotide or oligonucleotide as described herein. In some embodiments, Z may further comprise an arresting construct, which can slow or halt translocation of the polynucleotide.

Interactions with Interior of the Nanopore

Depending on the nature of the residues within the interior of the nanopore, speed control elements could include substituents that will interact with the residues, which are not limited to the following examples:
a) Negatively charged substituents such as COOH, PO₄³⁻, or SO₄²⁻;
b) Positive charged substituents such as quaternary ammonium, or protonated NH₂ moieties;
c) H-bonding substituents, such as NH₂, OH and SH moieties;
d) Pi-stacking substituents such as aromatic groups (phenyl, pyridine, imidazole, pyrrole);
e) Electrostatic interactions not limited to ionic and halogen bonding; Van der Waals interactions not limited to dipole-dipole and dipole-induced dipole; or
f) Fluorophilic interactions.

In some embodiments, the speed control element may include one or more moieties to increase non-covalent interaction with the interior of the nanopore. In some embodiments, the one or more moieties are selected from the group consisting of:

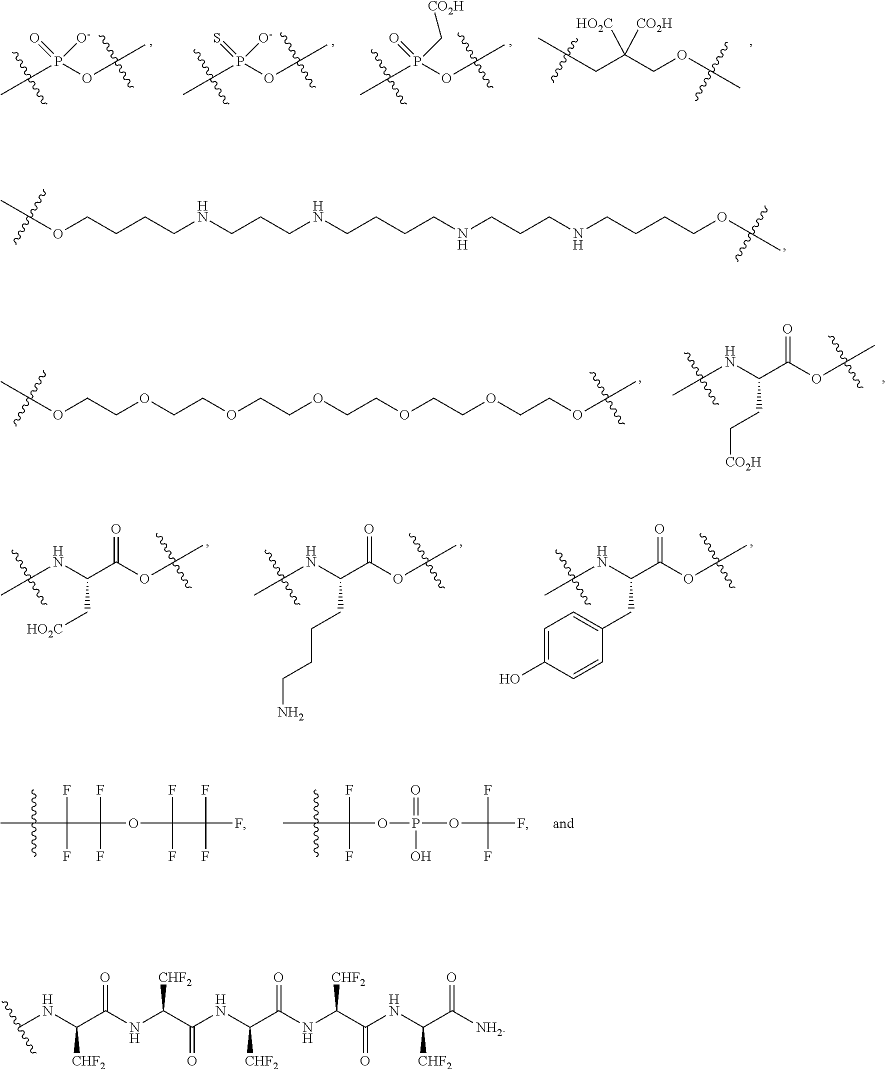

In some embodiments, the speed control element may be introduced through various means, such as attaching a phosphoramidite, spermine, lysine, glutamic acid, or tyrosine moiety through chemical synthesis/conjugation (e.g., Click chemistry, amine-NHS ester).

### Structural Configuration to Impeded Translocation

The nanopores for polynucleotide sequencing usually comprise a narrow channel to allow the translocation of polynucleotide. By imparting structural rigidity to the polynucleotide, the flexibility of the polynucleotide strand is reduced, slowing translocation.

There are several ways of introducing such structural configurational elements. In some embodiments, the one or more modifications introduce chirality to a non-bridging oxygen on the phosphate backbone. In one or more embodiments, the one or more modifications replaces phosphodiester linkages with phosphorothioate linkages. In some embodiments, there modifications introduce kinks to the oligonucleotide. In some embodiments, the oligonucleotide adopts slight distortion and reduces flexibility of the strand. A report by Baran et al. (Science 361, 2018, 1234) demonstrated stereoselective formation of the phosphorous centers.

In some embodiments, the modified nucleobase is selected from the group consisting of locked nucleic acid (LNA), constraint ethyl nucleic acid (cEtNA), and ethylene bridged nucleic acid (ENA).

### Voltage Dependent Speed Control Elements

One of the main factors contributing to the fast translocation of polynucleotide through a nanopore is the electrophoretic force exerted by an external applied electric field on the negatively charged polynucleotide strand. Reducing the charge density of the polynucleotide strand would result in lower electrophoretic force being exerted. In some embodiments, Z includes one or more moieties to alter or reduce the net charge of the modified nucleotide. In some embodiments, the one or more moieties are electrically neutral moieties. In some embodiments, the one or more moieties are selected from the group consisting of: spacer C3, spacer C12, spacer9, spacer18, and methylphosphonate. In some embodiments, the one or more moieties are positively charged moieties. In some embodiments, the one or more moieties are selected from the group consisting of: spermine phosphoramidite, arginine, lysine, and histidine amino acid derivatives.

In some embodiments, the one or more moieties are selected from the group consisting of:

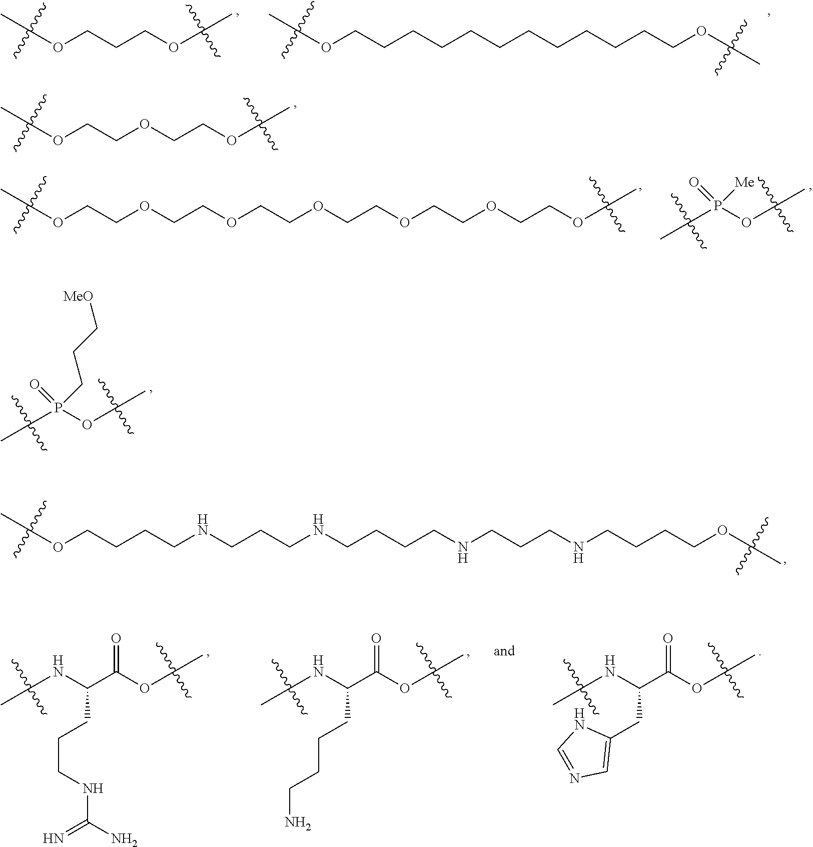

### Steric Dependent Speed Control Elements

In some embodiments, the speed control element includes one or more moieties to increase the bulk size of Z and increase steric hinderance. In some embodiments, the speed control element may be introduced through various means, such as attaching a phosphoramidite through chemical synthesis/conjugation (e.g., Click chemistry, amide coupling, thiol-maleimide coupling, disulfide formation).

In some embodiments, further functionalization would result in increased steric bulk, for example, by coupling appropriate NHS esters to amino groups, or a Cu-Click reaction between an alkyne and an azide, or thiol-maleimide coupling, or disulfide formation, or with affinity tags such as biotin-streptavidin and spytag-spycatcher.

In some embodiments, the one or more moieties for speed control element are selected from the group consisting of:

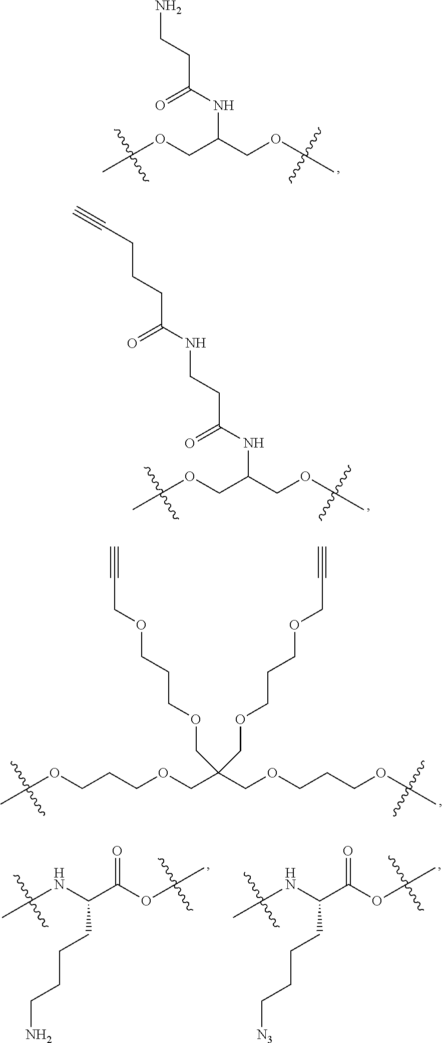 [SMILES: *OCC(CO*)NC(=O)CCN ; *OCC(CO*)NC(=O)CCNC(=O)CCCC#C ; *OCCCOCC(COCCCO*)(COCCCOCC#C)COCCCOCC#C ; *N[C@@H](CCCCN)C(=O)O* ; *N[C@@H](CCCCN=[N+]=[N-])C(=O)O*]

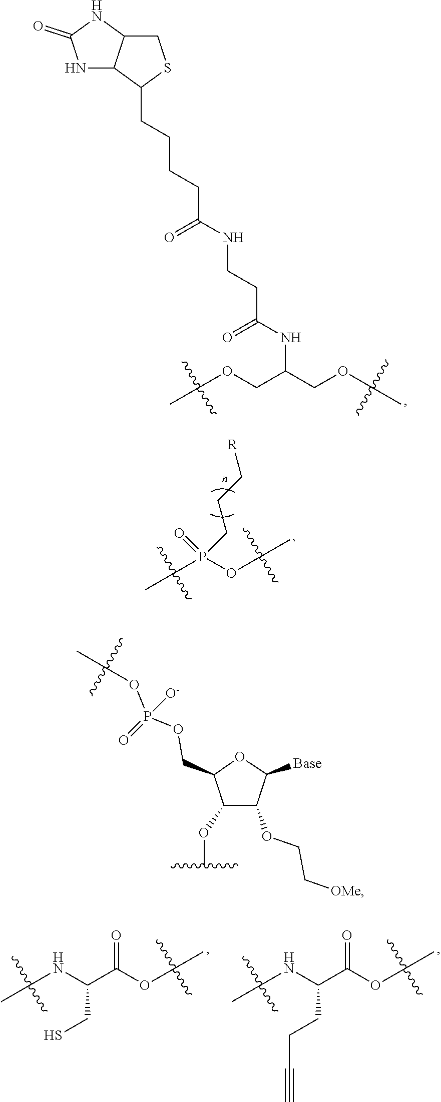

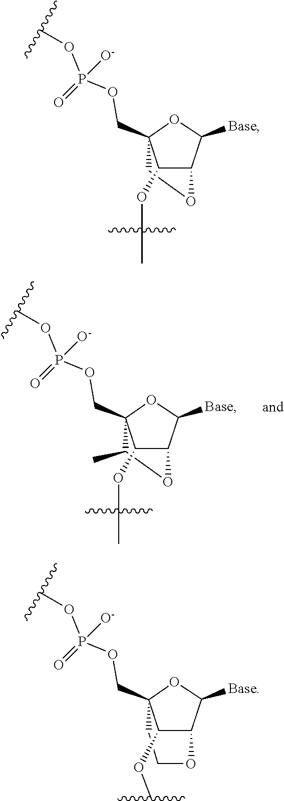

In some embodiments, peptide-based speed control elements are employed to modulate translocation. In some embodiments, steric bulk is introduced along the peptide based cyclic loop. In some embodiments, steric bulk is introduced by attaching a bulky group such as camphor or adamantyl. In some embodiments, steric bulk is introduced using kink-inducing amino acids such as proline and glycine. In some embodiments, steric bulk is introduced by deliberately introducing secondary structures such as α-helix or β-pleated sheet.

Modifying the charge density of various segments along the cyclic loop is easily achieved through a judicious selection of acidic or basic or neutral residues. Amino acids have long been known to interact with one another. In some embodiments, the peptide cyclic loop has varying types of interactions with the residues in the nanopore. In some embodiments, these interactions are electrostatic interactions between lysine and glutamic acid, hydrogen bonding, pi-stacking or Van der Waals interactions.

The design of the cyclic loop could involve a combination of various strategies to achieve tunable speed control, with the aim of ensuring high-accuracy base calling. In some embodiments, each of L1 and L2 independently comprises a conjugating moiety selected from the group consisting of amine-NHS ester, amine-imidoester, amine-pentofluorophenyl ester, amine-hydroxymethyl phosphine, carboxyl-carbodiimide, thiol-malcimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene.

Disclosed herein is a method for determining a sequence of a polynucleotide in a nanopore-based sequencing system, the method comprises providing a modified oligonucleotide, wherein the modified oligonucleotide comprises one or more modifications in at least one of a phosphate backbone, a nucleobases, or a sugar, wherein the one or more modifications impede translocation through a nanopore. In some embodiments, the method comprises applying a bias across a nanopore to cause the modified oligonucleotide to insert into and translocate through the nanopore. In some embodiments, the method comprises detecting and identifying a reporter moiety when the modified oligonucleotide passes through the nanopore. In another aspect, the method comprises detecting and identifying a base on the oligonucleotide when the oligonucleotide passes through the nanopore.

In some embodiments, the modified oligonucleotide comprises one or more modified nucleotides that comprise a cyclic loop, the cyclic loop having a first end attached to a first position of a nucleotide of the oligonucleotide and a second end attached to a second position of the nucleotide.

In some embodiments, the method further comprises cleaving a cleavable bond on the one or more modified nucleotides between the first and the second positions, thereby elongating the one or more modified nucleotides to form an elongated polymer, and (i) detecting and identifying a reporter moiety when the elongated polymer passes through the nanopore; or (ii) detecting and identifying a nucleobase on the one or more nucleotides when the elongated polymer passes through the nanopore.

In some embodiments, the cyclic loop comprises a first linking group, a second linking group, and a spacer between the first and the second linking groups.

In some embodiments, the spacer comprises a oligonucleotide or modified oligonucleotide or phosphoramidite analogs having 10 to 100 repeating units, polypeptide having 10 to 100 repeating units, alkyl chains having 5 to 50 carbons, hydrophilic polymers having 10 to 100 repeating units selected form the group consisting of polyethyleneglycol, polyvinyl alcohol, polyacrylamide, polyvinylpyrrolidone, polystyrenesulfonate, and polyethyleneimine, hydrophobic polymers having 10 to 100 repeating units selected from the group consisting of polylactic acid, polymethylmethacrylate, and polystyrene, and combinations thereof.

In some embodiments, the one or more modifications in at least one of a phosphate backbone, a nucleobases, or a sugar are isolated in a single region of the oligonucleotide.

In another aspect, the one or more modifications in at least one of a phosphate backbone, a nucleobase, or a sugar are interspersed between one or more modified nucleotides in the modified oligonucleotide.

Disclosed herein are kits for performing the disclosed methods as provided. The kit may comprise nucleotides or oligonucleotides with any of the modifications described in the present disclosure.

Disclosed herein are systems for determining a sequence of a polynucleotide using any of the methods described in the present disclosure. In another aspect, the polynucleotide comprises a plurality of oligonucleotides with any of the modifications described in the present disclosure.

Disclosed herein is a modified nucleotide, the modified nucleotide comprises a cyclic loop linking one or more atoms of the modified nucleotide, wherein the cyclic loop comprises a first linking group, a second linking group, and optionally, a spacer and reporter. In some embodiments, the spacer and reporter each comprise one or more of the following moieties: (a) polypeptides having 10 to 100 repeating units, (b) pseudopeptides having 10 to 100 repeating units, (c) hydrophilic polymers having 10 to 100 repeating units, and (d) hydrophobic polymers having 10 to 100 repeating units.

In some embodiments, the cyclic loop further comprises nucleosidic moieties, non-nucleosidic moieties, and/or peptide moieties.

In some embodiments, the modified nucleotide further comprises one or more of the following: backbone modification(s), sugar modification(s), and base modification(s) configured to reduce the translocation speed of the modified nucleotide through a nanopore having a constriction zone diameter less than 5 nm in width.

Disclosed herein is an oligonucleotide strand, the oligonucleotide strand comprises any of the modified nucleotides described in the present disclosure.

### EXAMPLES

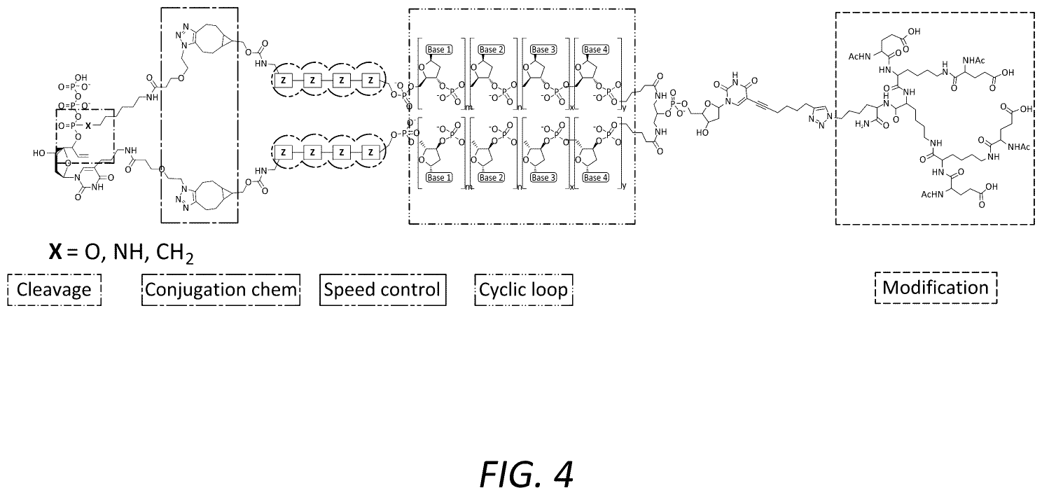

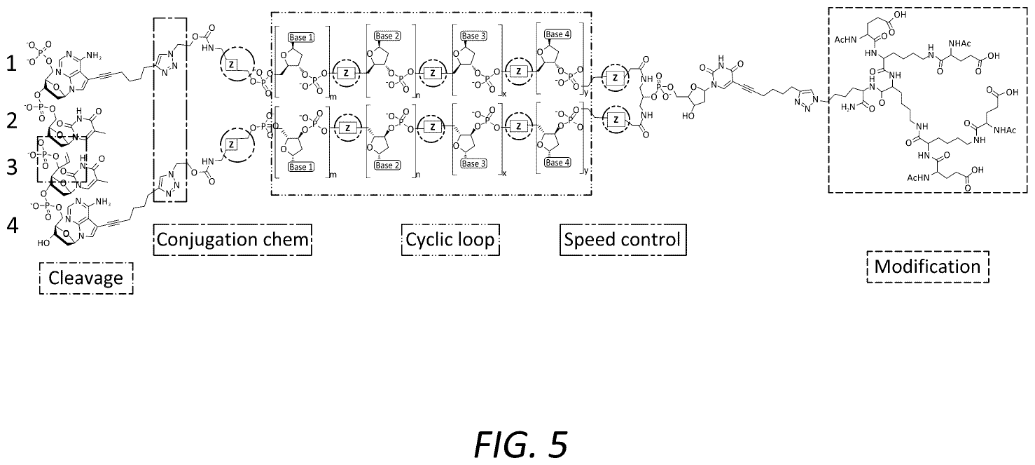

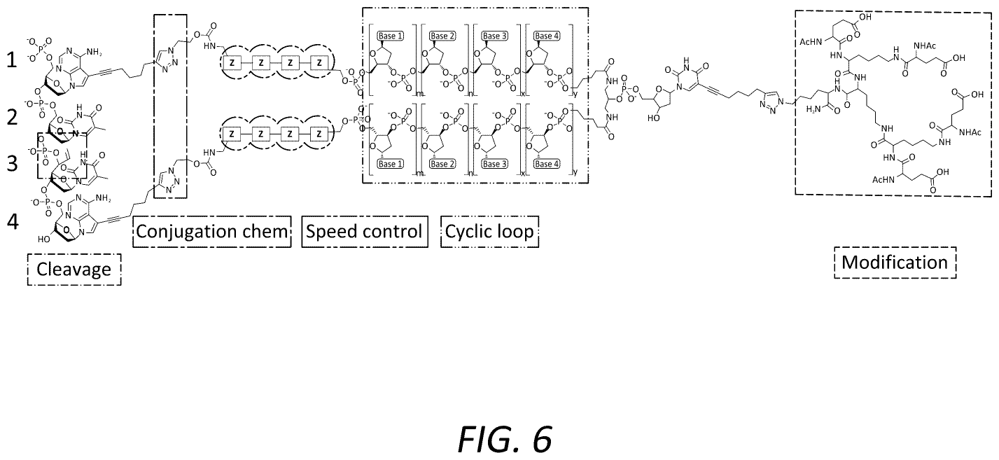

In some embodiments, a modified nucleotide comprises a speed control element interspersed in a cyclic loop (FIG. 3). In some embodiments, a modified nucleotide comprises a speed control element isolated within a specific region of a cyclic loop (FIG. 4). In some embodiments, a modified k-mer comprises a speed control element interspersed in a cyclic loop (FIG. 5). In some embodiments, a modified k-mer comprises a speed control element isolated within a specific region of a cyclic loop (FIG. 6).

In some embodiments, the one or more moieties comprises

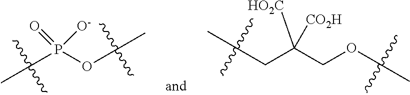 [SMILES: *OP(*)(=O)[O-] ; *CC(CO*)(C(=O)O)C(=O)O]

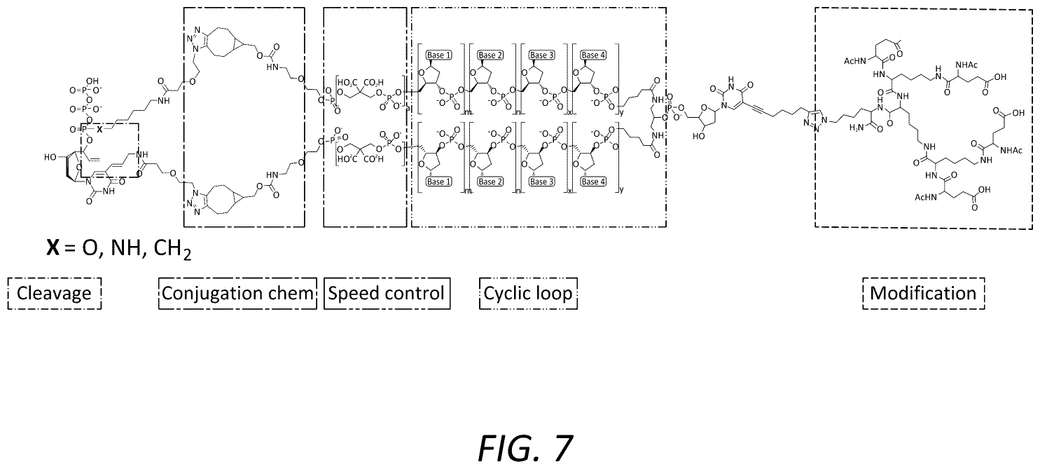

### (FIG. 7).

In some embodiments, the one or more moieties is

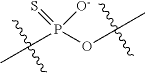 [SMILES: *OP(*)([O-])=S]

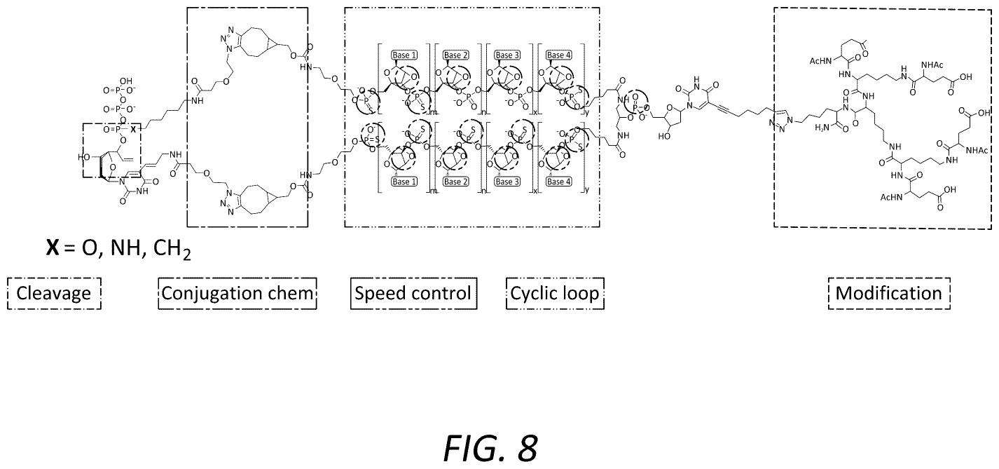

and the nucleotide comprises locked nucleic acid modifications (FIG. 8).

In some embodiments, the one or more moieties are

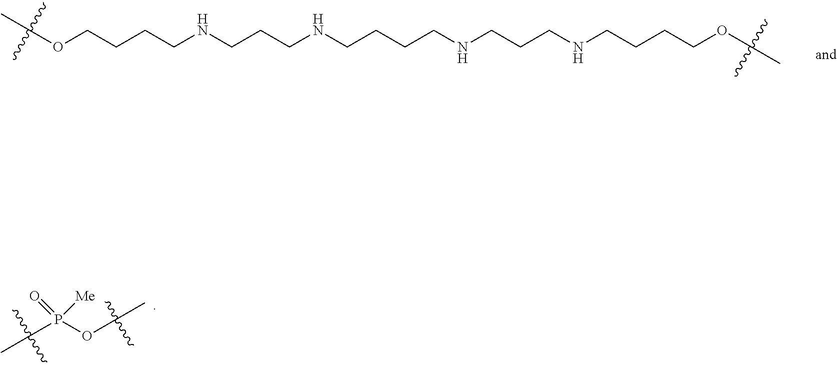 [SMILES: *OCCCCNCCCNCCCCNCCCNCCCCO* ; *OP(*)(C)=O]

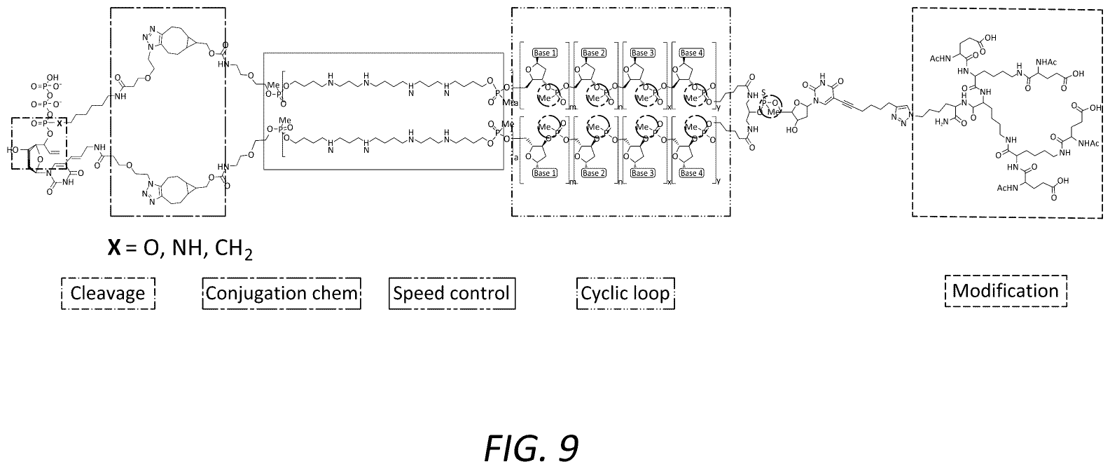

### (FIG. 9).

In some embodiments, the one or more moieties is

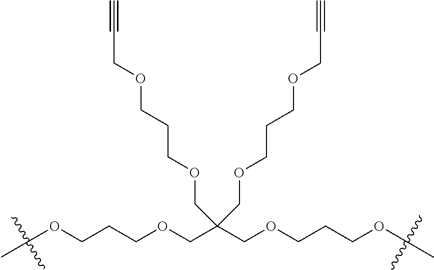 [SMILES: *OCCCOCC(COCCCO*)(COCCCOCC#C)COCCCOCC#C]

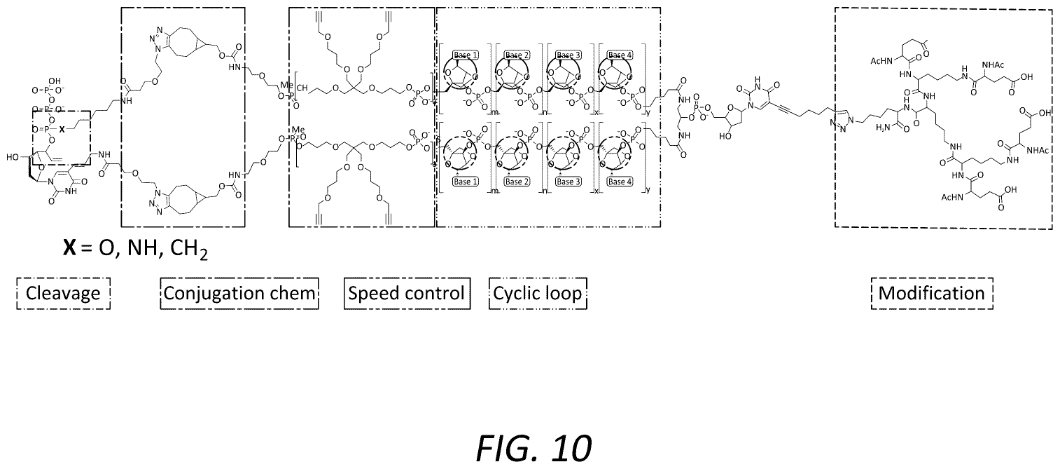

and the nucleotide comprises locked nucleic acid modifications (FIG. 10).

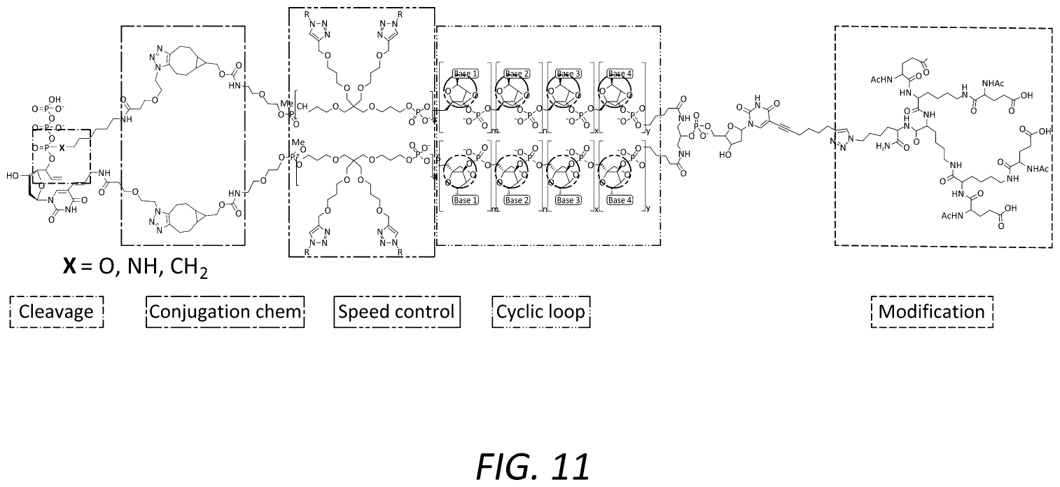

In some embodiments, the alkyne on the one or more moieties is further functionalized via Cu-click to increase steric bulk along the cyclic loop (FIG. 11).

What is claimed is:

1. A modified oligonucleotide comprising one or more modifications in at least one of a phosphate backbone, a nucleobase, or a sugar, wherein the one or more modifications impede translocation through a nanopore.

2. The modified oligonucleotide of claim 1, wherein the one or more modifications are synthetic moieties to:
a) increase non-covalent interactions with an interior of the nanopore;
b) increase the bulk size of the oligonucleotide;
c) alter the charge density of the oligonucleotide;
d) increase the steric hinderance of the oligonucleotide; and/or
e) increase non-covalent interactions with a lipid bilayer that supports the nanopore.

3. The modified oligonucleotide of claim 1, wherein the one or more modifications replaces a non-bridging oxygen on the phosphate backbone and is selected from the group consisting of:

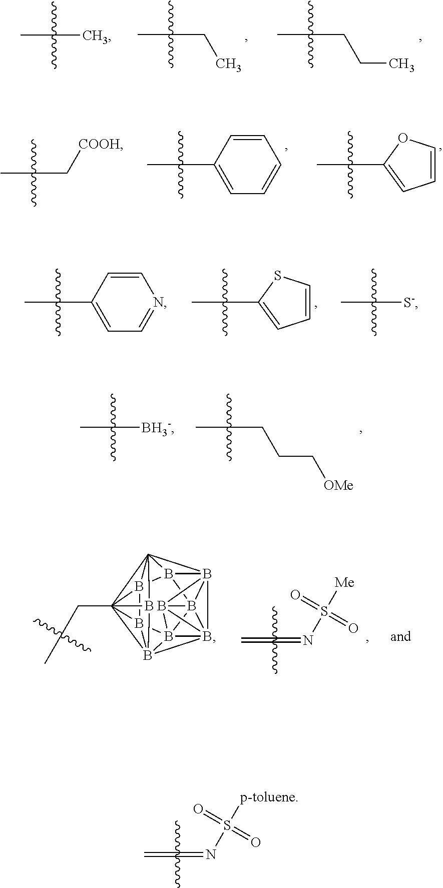

4. The modified oligonucleotide of claim 1, wherein the one or more modifications introduces chirality to a non-bridging oxygen on the phosphate backbone.

5. The modified oligonucleotide of claim 1, wherein the one or more modifications replaces a single bridging oxygen in the phosphate backbone with one or more elements selected from O, S, and —NH—.

6. The modified oligonucleotide of claim 1, wherein the one or more modifications comprises a substitution on the phosphate backbone with one or more nucleotide linkers to produce a modified oligonucleotide having one of the following nucleotide linking structures:

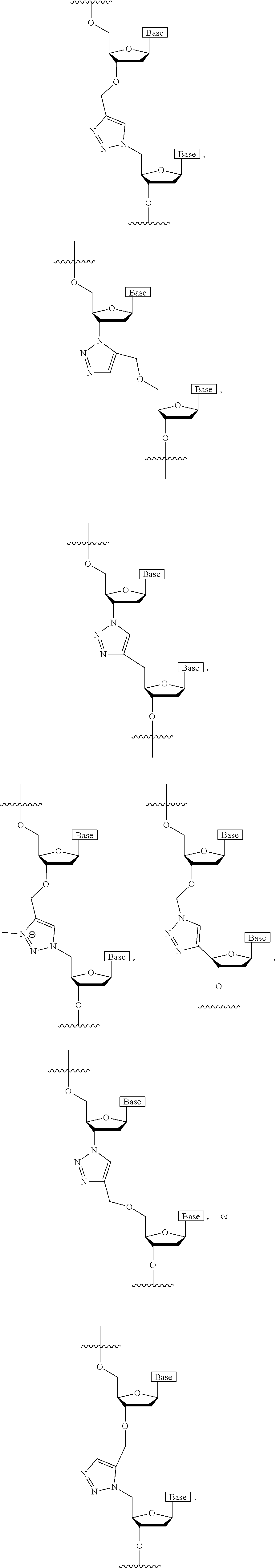

7. The modified oligonucleotide of claim 1, wherein the one or more modifications comprises a modified sugar structure and the oligonucleotide contains a modified nucleotide selected from the group consisting of:

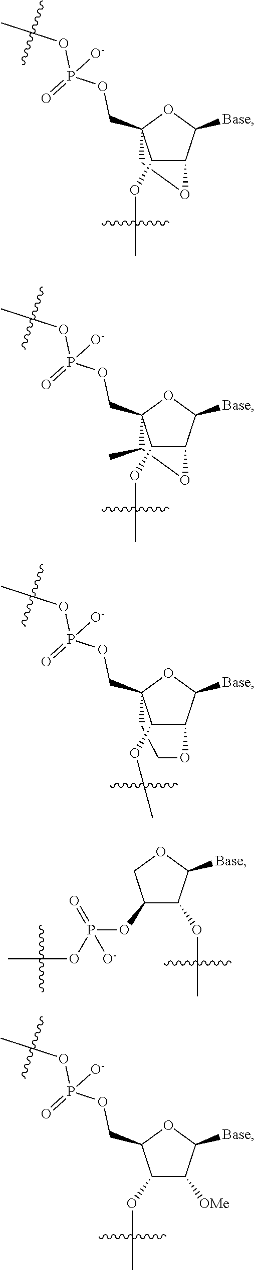

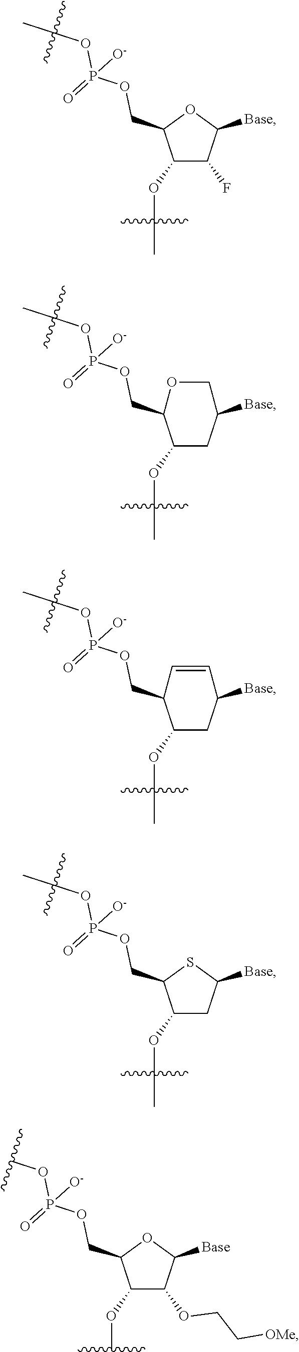

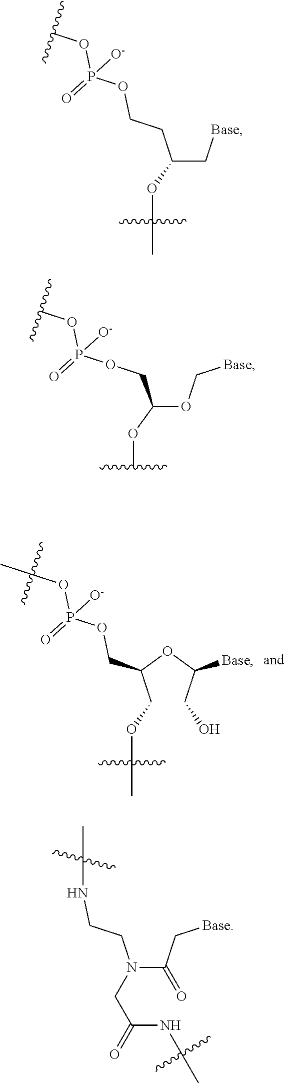

8. The modified oligonucleotide of claim 1, wherein the one or more modifications comprises a moiety attached to the nucleobase and the moiety is selected from the group consisting of: 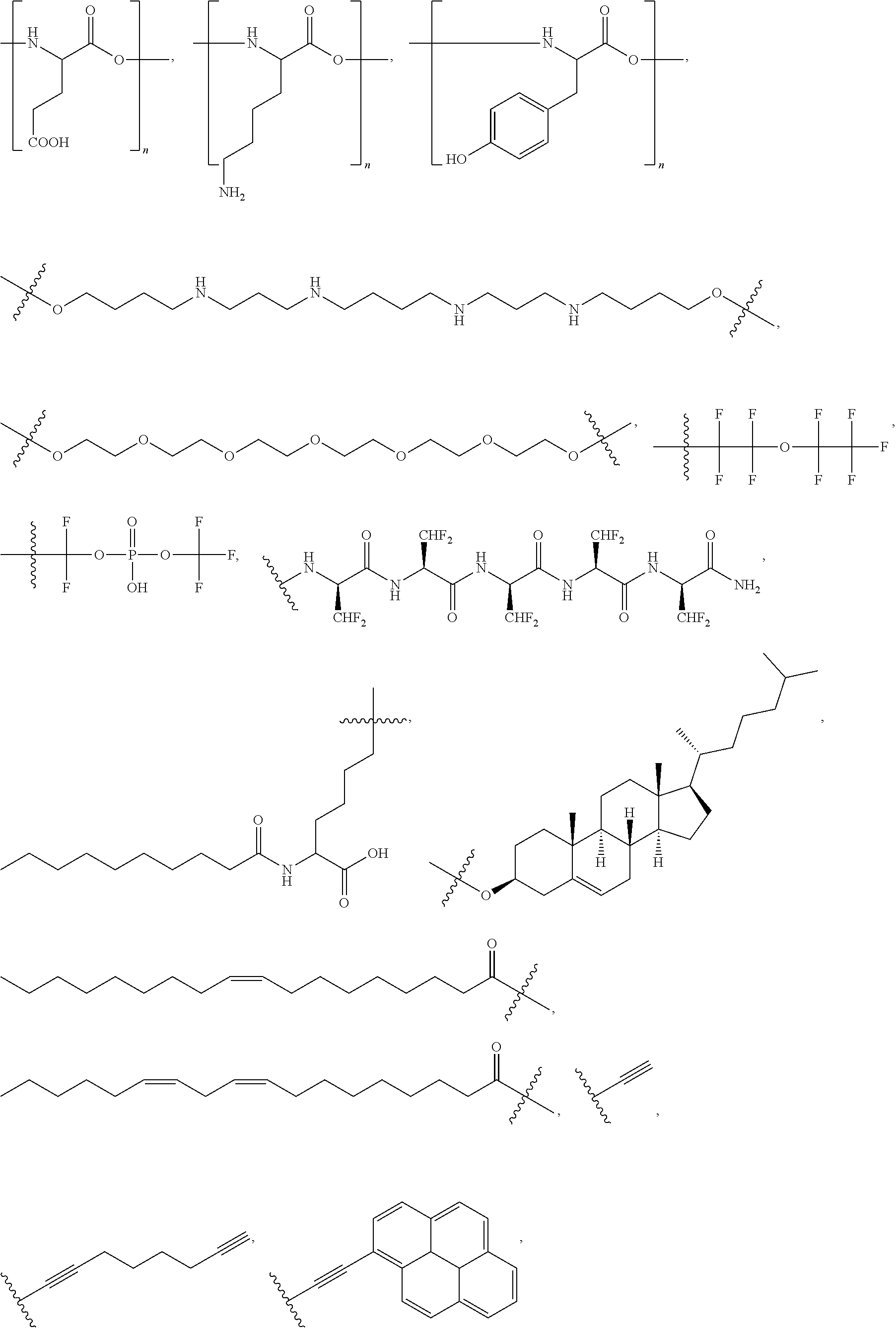 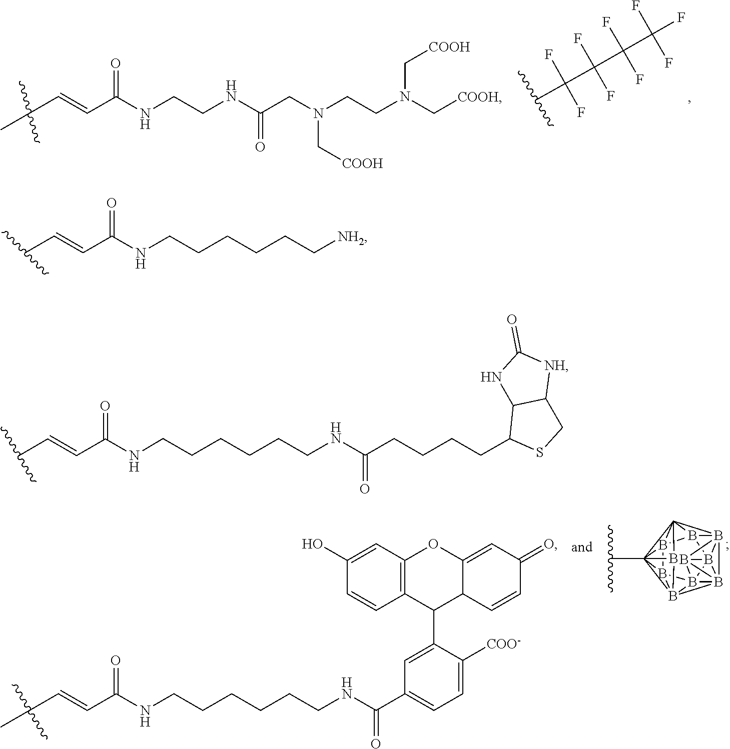
wherein:
n is a positive integer from 1 to 100.

9. The modified oligonucleotide of claim 1, wherein the modified nucleobase is selected from the group consisting of:

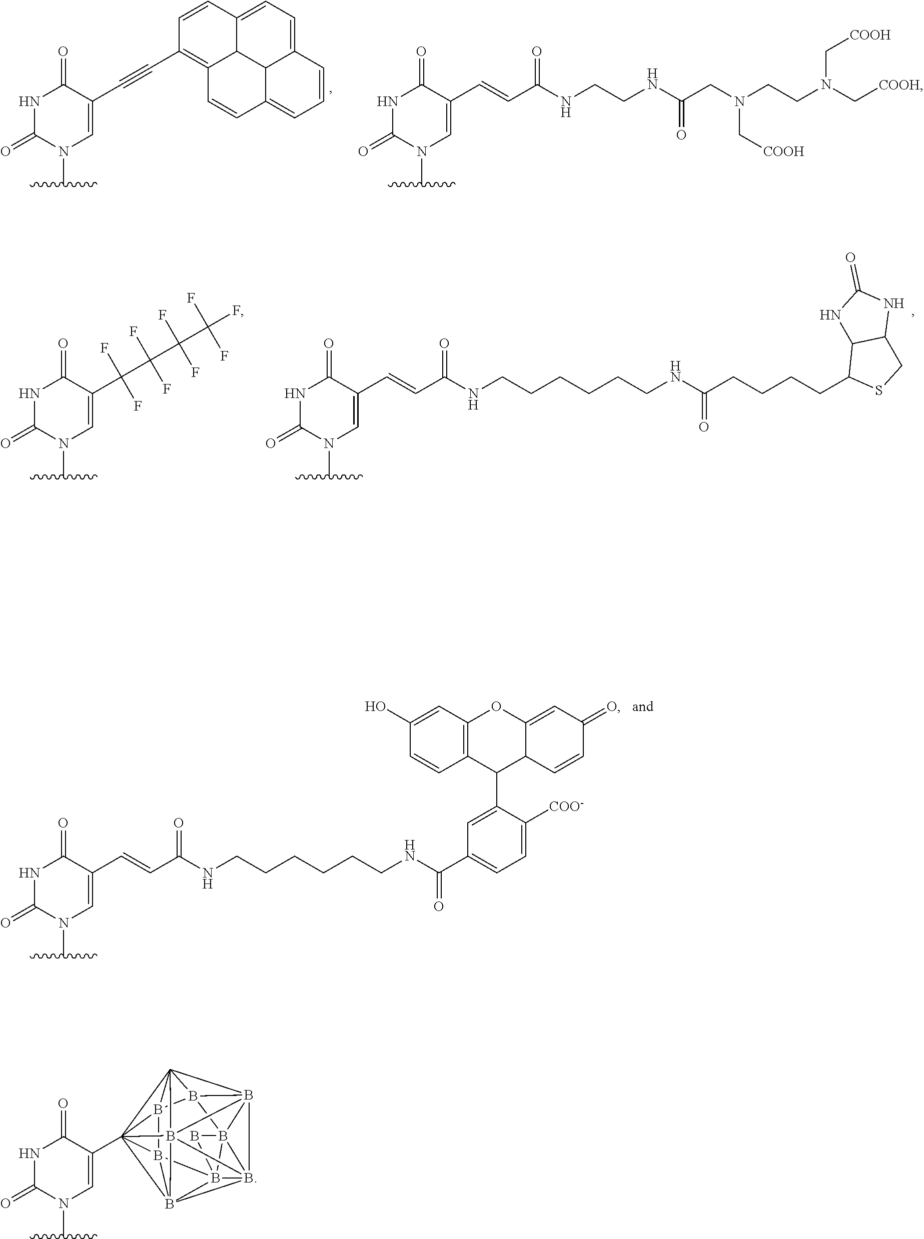

10. The modified oligonucleotide of claim 1, wherein the nucleobase comprises a nucleobase modification including one or more of: glutamic acid, polyglutamic acid, polylysine, polyamines, fluorine, G-quadraplexes, DNA origami, or macrocycles.

11. The modified oligonucleotide of claim 1, wherein the modified oligonucleotide additionally comprises a modified nucleotide containing a cyclic loop bridging the nucleobase and the phosphate backbone or bridging one portion of the phosphate backbone and an oxygen on the phosphate backbone.

12. A modified nucleotide having one of the following structures: 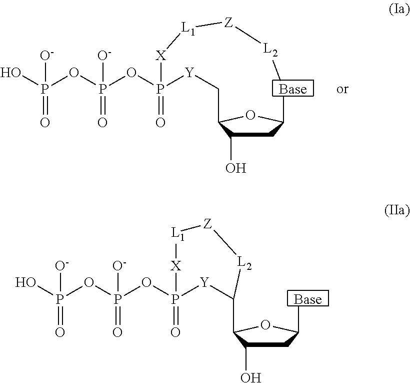
wherein:
X is —O—, —CH₂—, or —NH—;
Y is —O—, —S—, or —NH—;
Base is a nucleobase;
L₁ is a first linking group;
L₂ is a second linking group; and
Z is a cyclic loop modification containing at least one moiety to reduce, slow, or halt translocation of the nucleotide through a nanopore.

13. The modified nucleotide of claim 12, wherein Z includes a spacer and reporter, wherein the spacer and reporter each comprises one or more of the following moieties:
a) polypeptides having 10 to 100 repeating units
b) pseudopeptides having 10 to 100 repeating units
c) hydrophilic polymers having 10 to 100 repeating units
d) hydrophobic polymers having 10 to 100 repeating units.

14. The modified nucleotide of claim 12, wherein Z includes one or more moieties to increase non-covalent interaction with the interior of the nanopore.

15. The modified nucleotide of claim 14, wherein the one or more moieties are selected from the group consisting of:

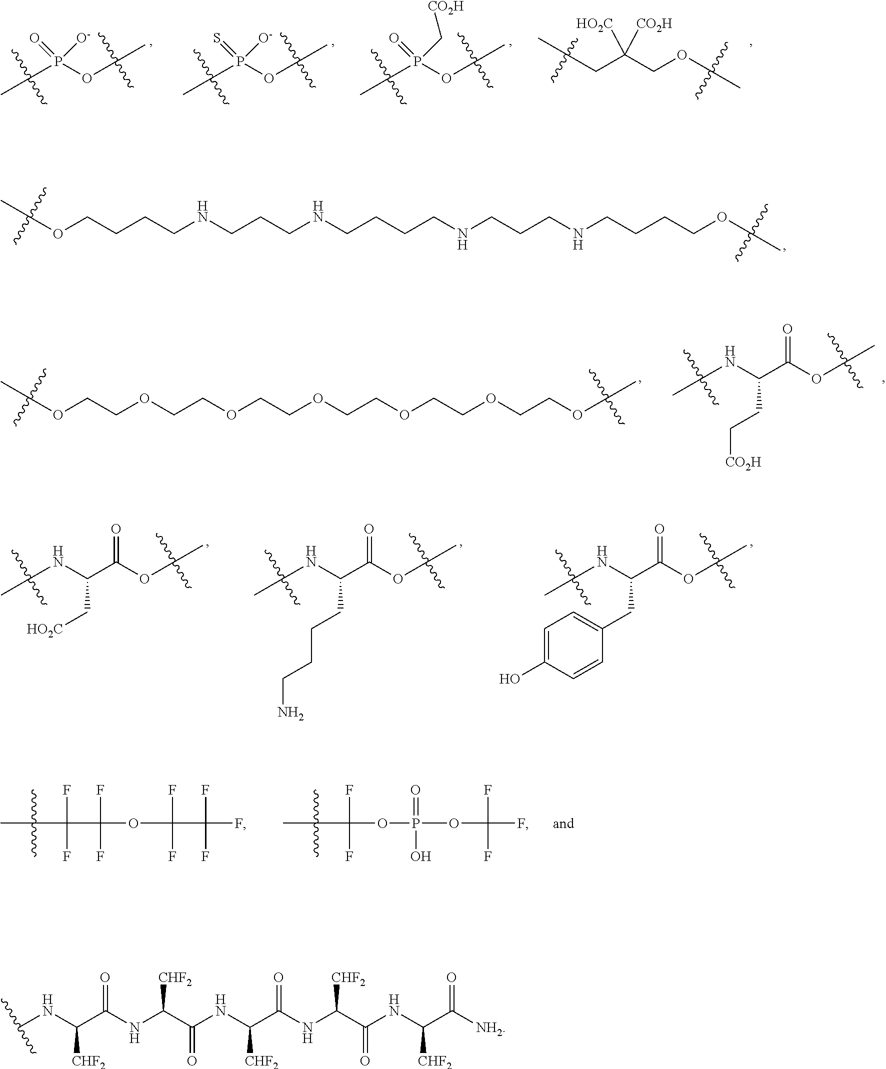

16. The modified nucleotide of claim 12, wherein Z includes one or more moieties to alter or reduce the net charge of the modified nucleotide.

17. The modified nucleotide of claim 16, the one or more moieties are selected from the group consisting of:

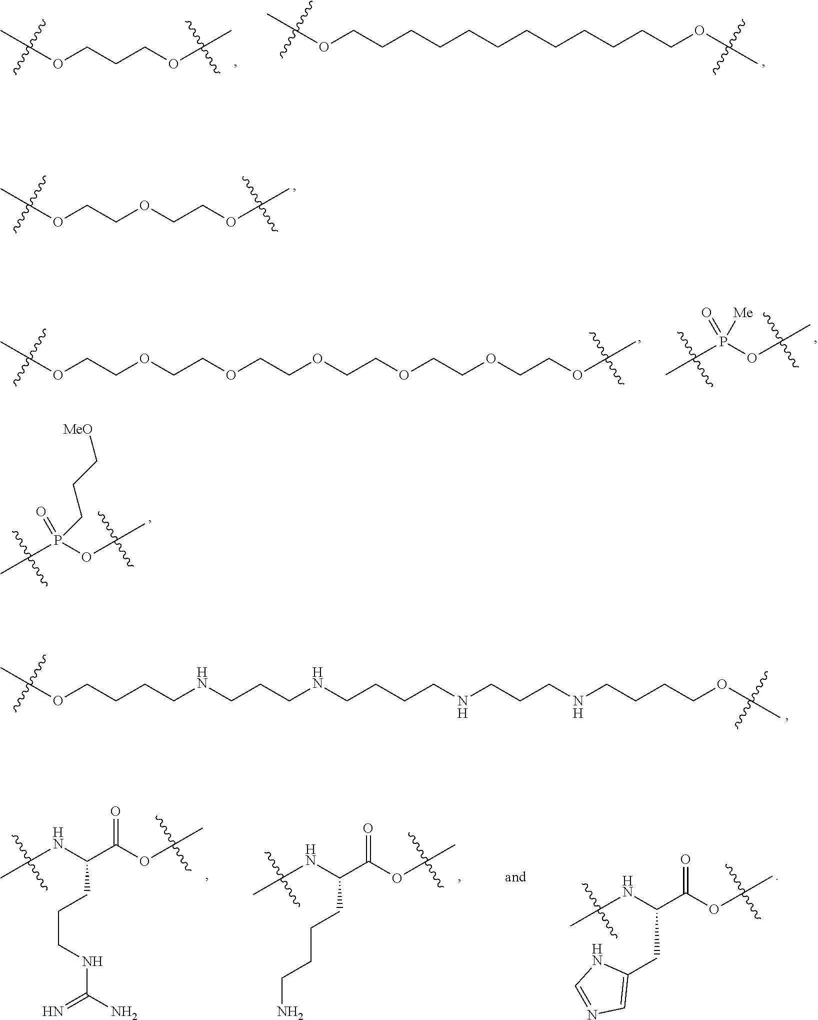

18. The modified nucleotide of claim 12, wherein Z includes one or more moieties to increase the bulk size of Z and increase steric hinderance.

19. The modified nucleotide of claim 18, wherein the one or more moieties are selected from the group consisting of:

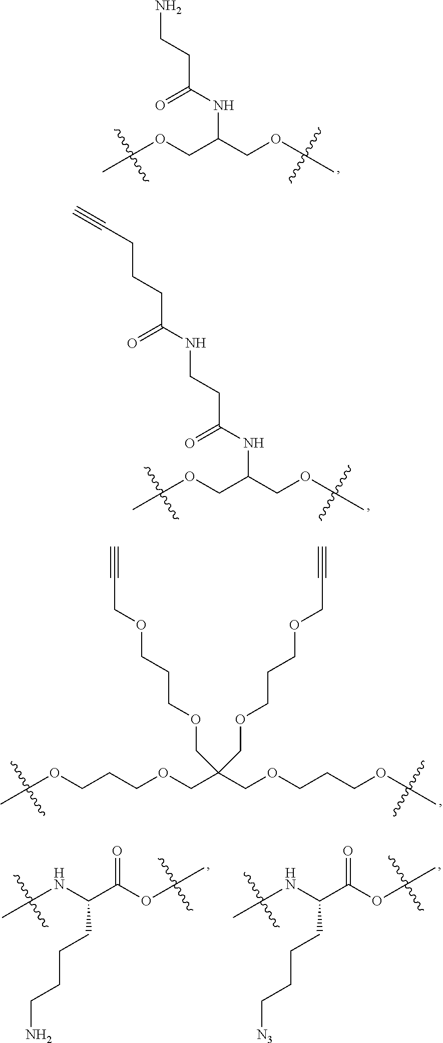 [SMILES: *OCC(CO*)NC(=O)CCN ; *OCC(CO*)NC(=O)CCNC(=O)CCCC#C ; *OCCCOCC(COCCCO*)(COCCCOCC#C)COCCCOCC#C ; *N[C@@H](CCCCN)C(=O)O* ; *N[C@@H](CCCCN=[N+]=[N-])C(=O)O*]

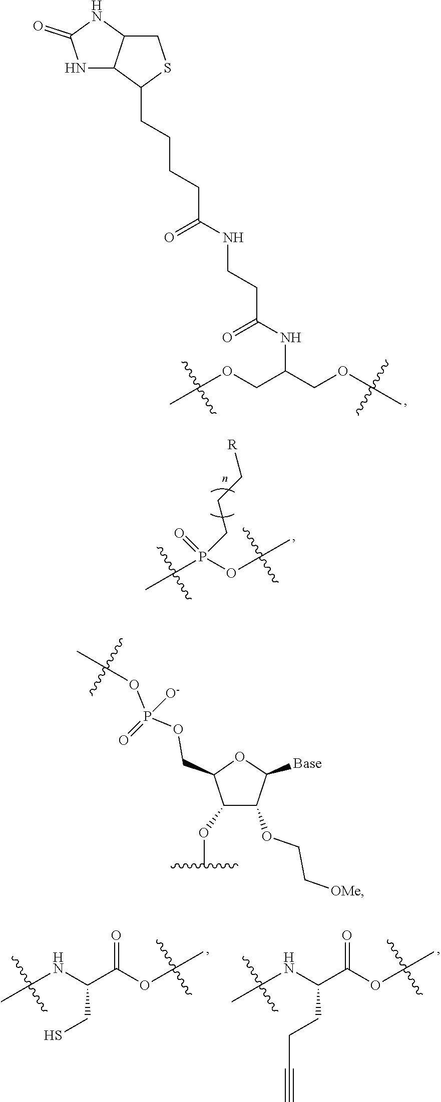

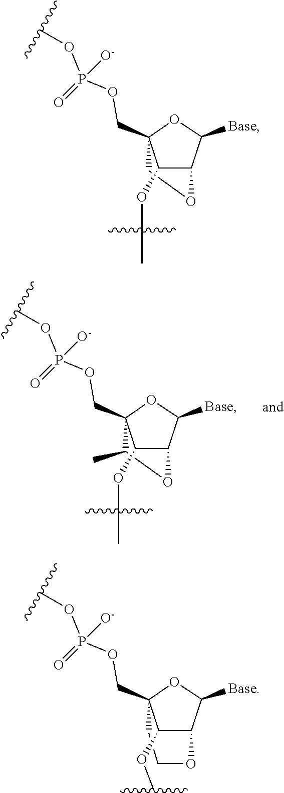

20. The modified nucleotide of claim 12, wherein each of L₁ and L₂ independently comprises a conjugating moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene.

21. A method for determining a sequence of a polynucleotide in a nanopore-based sequencing system, the method comprising:
providing a modified oligonucleotide comprising one or more modifications in at least one of a phosphate backbone, a nucleobases, or a sugar, wherein the one or more modifications impede translocation through a nanopore;
applying a bias across a nanopore to cause the modified oligonucleotide to insert into and translocate through the nanopore; and
(i) detecting and identifying a reporter moiety when the modified oligonucleotide passes through the nanopore; or
(ii) detecting and identifying a base on the oligonucleotide when the oligonucleotide passes through the nanopore.

22. The method of claim 21, wherein the modified oligonucleotide comprises one or more modified nucleotides that comprise a cyclic loop, the cyclic loop having a first end attached to a first position of a nucleotide of the oligonucleotide and a second end attached to a second position of the nucleotide.

23. The method of claim 22, further comprising cleaving a cleavable bond on the one or more modified nucleotides between the first and the second positions, thereby elongating the one or more modified nucleotides to form an elongated polymer; and
i. detecting and identifying a reporter moiety when the elongated polymer passes through the nanopore; or
ii. detecting and identifying a nucleobase on the one or more nucleotides when the elongated polymer passes through the nanopore.

24. The method of claim 23, wherein the cyclic loop comprises a first linking group, a second linking group, and a spacer between the first and the second linking groups.

25. The method of claim 24, wherein the spacer comprises a oligonucleotide or modified oligonucleotide or phosphoramidite analogs having 10 to 100 repeating units, polypeptide having 10 to 100 repeating units, alkyl chains having 5 to 50 carbons, hydrophilic polymers having 10 to 100 repeating units selected form the group consisting of polyethyleneglycol, polyvinyl alcohol, polyacrylamide, polyvinylpyrrolidone, polystyrenesulfonate, and polyethyleneimine, hydrophobic polymers having 10 to 100 repeating units selected from the group consisting of polylactic acid, polymethylmethacrylate, and polystyrene, and combinations thereof.

26. The method of claim 21, wherein the one or more modifications in at least one of a phosphate backbone, a nucleobases, or a sugar are isolated in a single region of the oligonucleotide.

27. The method of claim 21, wherein the one or more modifications in at least one of a phosphate backbone, a nucleobase, or a sugar are interspersed between one or more modified nucleotides in the modified oligonucleotide.

28. A kit for performing a method for determining a sequence of a polynucleotide in a nanopore-based sequencing system, the kit comprising the modified oligonucleotide according to claim 1.

29. A system for determining a sequence of a polynucleotide, the system configured to perform a method according to claim 21.

30. A system for performing a method for determining a sequence of a polynucleotide comprising a plurality of oligonucleotides, wherein the oligonucleotides are selected from any of the modified oligonucleotides according to claim 1.

31. A modified nucleotide comprising a cyclic loop linking one or more atoms of the modified nucleotide, wherein the cyclic loop comprises a first linking group, a second linking group, and optionally, a spacer and reporter, wherein the spacer and reporter each comprises one or more of the following moieties:
a) polypeptides having 10 to 100 repeating units;
b) pseudopeptides having 10 to 100 repeating units;
c) hydrophilic polymers having 10 to 100 repeating units;
d) hydrophobic polymers having 10 to 100 repeating units;
wherein the cyclic loop further comprises nucleosidic moieties, non-nucleosidic moieties, and/or peptide moieties.

32. The modified nucleotide of claim 31, further comprising one or more of the following: backbone modification(s), sugar modification(s), and base modification(s) configured to reduce the translocation speed of the modified nucleotide through a nanopore having a constriction zone diameter less than 5 nm in width.

33. An oligonucleotide strand comprising the modified nucleotide of claim 31.
